(19) United States
(12) Patent Application Publication
(10) Pub. No.: US 2017/0349949 A1
(43) Pub. Date: Dec. 7, 2017

(54) METHODS OF SELECTING SUBJECTS FOR TREATMENT WITH METABOLIC MODULATORS

(71) Applicant: Partikula LLC, Sunrise, FL (US)

(72) Inventor: David Kolb, Sunrise, FL (US)

(21) Appl. No.: 15/609,992

(22) Filed: May 31, 2017

Related U.S. Application Data
(60) Provisional application No. 62/404,564, filed on Oct. 5, 2016.
(60) Provisional application No. 62/345,545, filed on Jun. 3, 2016.

(51) Int. Cl. C12Q 1/68

(52) U.S. Cl. CPC C12Q 1/6883; C12Q 2600/172; C12Q 2600/106; C12Q 2600/156

(57) ABSTRACT

Methods of selecting a subject with cancer for treatment with an active agent that modifies pyruvate metabolism, the TCA cycle, or oxidative phosphorylation, as well as methods of treating the subject, determining the efficacy of the treatment, and adjusting the treatment dosage and frequency are provided. Methods of selecting and treating as subject typically include, (a) detecting the level of one or more biomarkers selected from the group consisting of one or more Mitochondrial Pyruvate Carriers (MPC), one or more components of the Pyruvate Dehydrogenase Complex (PDC), or mitochondrial glutamine transporter in diseased or disordered cells obtained from the subject; and (b) selecting the subject for treatment if the subject meets certain criteria and (c) administering the subject an effective amount of an active agent that modifies pyruvate metabolism, the TCA cycle, a related metabolic pathway, or oxidative phosphorylation to treat the disease or disorder.

### CROSS REFERENCE TO RELATED APPLICATIONS

This application claims priority to and benefit of U.S. Provisional Application No. 62/404,564, filed Oct. 5, 2016, and U.S. Provisional Application No. 62/345,545, filed Jun. 3, 2016, the disclosures of which are hereby incorporated herein by reference in their entirety.

### REFERENCE TO SEQUENCE LISTING

The Sequence Listing submitted as a text file named “PAR_101_ST25.txt,” created on May 31, 2017, and having a size of 6,936 bytes is hereby incorporated by reference pursuant to 37 C.F.R. §1.52(e)(5).

### FIELD OF THE INVENTION

The field of the invention generally relates to methods for selecting and treating subjects with a metabolic modulator, particularly a modulator that acts downstream of pyruvate import into the mitochondria.

### BACKGROUND OF THE INVENTION

Most differentiated mammalian cells direct pyruvate into mitochondria where it is oxidized for efficient ATP production. Many cancer cells, however, can divert pyruvate and its precursors to fuel other anabolic processes or convert it to lactate for excretion from the cell via a metabolic adaptation referred to as the Warburg effect (process reviewed in Schell, et al., Molecular Cell, 56:400-413 (2014), and references cited therein). Although several mechanisms contribute to this metabolic alteration, the synthesis and metabolism of pyruvate play an important and prominent role, and altered pyruvate metabolism appears to be particularly important in enabling and promoting the transformed phenotype in many cancers. In diseases outside of cancer there are often metabolic issues that lead to dysfunction and eventual cell death, as is the case in neurodegenerative disease, cardiomyopathies and other chronic illnesses. Unlike cancer, in these cases metabolism might be underperforming or there might be other issues that are preventing cells from meeting their energetic requirements. In these cases pyruvate and other metabolites and their pathways play an important role.

First, the synthesis of pyruvate in glycolysis is catalyzed by pyruvate kinase. Cancer cells can express a partially inhibited splice variant of pyruvate kinase (PK-M2), leading to decreased pyruvate production (Christofk, Nature 452, 230-233 (2008), Christofk, Nature, 452, 181-186 (2008); Luo and Semenza, Oncotarget, 2, 551-556 (2011); Yang et al., Nature, 480, 118-122 (2011); Yeh et al., Oncol. Rep., 19, 81-91 (2008). Second, lactate dehydrogenase A (LDHA) and the monocarboxylate transporters MCT 1-4, the two protein classes that mediate pyruvate conversion to lactate and its export, are often upregulated in cancer cells leading to decreased pyruvate oxidation (Azuma et al., Pharmacogenomics, 8, 1705-1713 (2007); Le Floch et al., Proc. Natl. Acad. Sci. USA 108, 16663-16668 (2011); Gotanda et al., Anticancer Res., 33, 2941-2947 (2013); Koukourakis et al., Br. J. Cancer, 89, 877-885 (2003); Pinheiro et al., Virchows Arch. 452, 139-146 (2008)). Third, the enzymatic step following mitochondrial import is the conversion of pyruvate to acetyl-CoA by the pyruvate dehydrogenase (PDH) complex (also referred to as PDC). Cancer cells frequently exhibit increased expression of PDH kinases PDK 1-4, which phosphorylate and inactivate PDH (Kim et al., Cell Metab. 3, 177-185, (2006); McFate et al., J. Biol. Chem., 283, 22700-22708, (2008)). This PDH regulatory mechanism is important for oncogene-induced transformation and reversed in oncogene-induced senescence (Kaplon et al., Nature, 498, 109-112 (2013)).

Pyruvate dehydrogenase kinase (PDK) inhibitors such as dichloroacetate can reduce or prevent the phosphorylation that inactivates PDH in cancer cells, thereby driving an increase in conversion of pyruvate to acetyl-CoA, shifting the metabolism of cancer cells from glycolysis to glucose oxidation and reversing the suppression of mitochondria-dependent apoptosis (Sutendra, et al., Front Oncol., 3:38 (2013)). The treatment also correlates with an increase in pyruvate oxidation (Michelakis et al., Sci. Transl. Med. 2, 31ra34, (2010)).

Conversely, the Inverse Warburg effect occurs when metabolic reprogramming leads to the up-regulation of oxidative phosphorylation (OXPHOS) in mitochondria of certain cells. The Inverse Warburg effect has been characterized as a compensatory increase in OXPHOS designed to maintain adequate energy production, and has been identified a hallmark of neurodegenerative disease progression and a complication of diabetes (Demetrius, et al., Biogerontology, 13(6):583-94 (2012), Demetrius, et al., Front Physiol, 5: 522 (2015), Craft, et al., Arch Neurol. 69(1):29-38 (2012)). Additionally, oxidative phosphorylation (OXPHOS) regulates apoptosis through the OXPHOS complexes (i.e., I, II, III, IV, and V) (Yadav, et al., Cell Death and Disease, 6, e1969; doi:10.1038/cddis.2015.305 (2015)). Thus, such treatment strategies that, for example, increase PDK can be used to divert cellular metabolism toward aerobic glycolysis, increase cellular longevity, or a combination thereof.

However, there remains a need to determine if these treatment strategies will be effective in a subject in need thereof prior to initiating treatment.

It is an object of the invention to provide methods of determining if active agents that modify pyruvate metabolism; the tricarboxylic acid (TCA) cycle; citrate transport or another transporter or enzyme related to formation or cycling of malate, citrate, or acetyl-CoA; glutaminolysis or a transporter or enzyme associated therewith such as glutaminase; or oxidative phosphorylation will be effective for treating subjects in need thereof.

It is a further object of the invention to provide strategies for selecting subjects and treating them with one or more active agents that modify pyruvate metabolism, the tricarboxylic acid (TCA) cycle, or oxidative phosphorylation, as well as monitoring efficacy, adjusting dosage, and discontinuing treatment when necessary.

### SUMMARY OF THE INVENTION

Methods of selecting a subject in need thereof for treatment with an active agent that modifies pyruvate metabolism; the TCA cycle; citrate transport or another transporter or enzyme related to formation or cycling of malate, citrate, or acetyl-CoA; glutaminolysis or a transporter or enzyme associated therewith such as glutaminase; or oxidative phosphorylation, as well as methods of treating the subject, determining the efficacy of the treatment, and adjusting the treatment dosage and frequency are provided.

Methods of selecting and treating a subject typically include, (a) detecting the level of one or more biomarkers selected from the group consisting of one or more Mitochondrial Pyruvate Carriers (MPC), one or more components of the Pyruvate Dehydrogenase Complex (PDC), mitochondrial glutamine transporter, or a combination thereof in diseased or disordered cells obtained from the subject; and (b) selecting the subject for treatment if the subject meets certain criteria that can include the diseased and disordered cells having a level of the biomarker of at least 20%, 25%, 30%, 35%, 40%, 45%, 50%, 55%, 60%, 65%, 70%, 75%, 80%, 85%, 90%, 95%, 100%, or more than 100% relative to a control. Selected subjects can be (c) administered an effective amount of an active agent that modifies pyruvate metabolism; the TCA cycle; citrate transport or another transporter or enzyme related to formation or cycling of malate, citrate, or acetyl-CoA; glutaminolysis or a transporter or enzyme associated therewith such as glutaminase; or oxidative phosphorylation to treat the disease or disorder.

Although in preferred embodiments, the biomarker(s) is Mitochondrial Pyruvate Carrier 1, Mitochondrial Pyruvate Carrier 2, or the combination thereof, additional biomarkers include glutamine transporter, components of the PDC such as pyruvate dehydrogenase subunit a, pyruvate dehydrogenase subunit β, dihydrolipoyl transacetylase, dihydrolipoyl dehydrogenase, and combinations thereof. In some embodiments, the levels of 2, 3, 4, 5, or more biomarkers are determined.

In some embodiments, subjects that would not be selected due to low or absent expression of MPC1 and/or MPC2 are nonetheless selected if the level of glutamine transporter is not substantially lower, is equal to, or is greater than a control. Similarly, in some embodiments, subjects that would not be selected due to low or absent expression of glutamine transporter are nonetheless selected if the level of MPC is not substantially lower, is equal to, or is greater than a control.

Preferred active agents for treating the subject are also provided and include, for example, modulators of pyruvate dehydrogenase kinase; the tricarboxylic acid (TCA) cycle; citrate transport or another transporter or enzyme related to formation or cycling of malate, citrate, or acetyl-CoA; glutaminolysis or a transporter or enzyme associated therewith such as glutaminase; and the electron transport chain. The methods can be utilized to modulate the metabolism of diseased or disordered cells in the subject. For example, if the disease or disorder is characterized by cells exhibiting a Warburg effect metabolic phenotype, and the active agent can be one that shifts the metabolism of the cells from glycolysis to glucose oxidation, reverses the suppression of mitochondria-dependent apoptosis, increases the oxidation of pyruvate, reduces the conversion of pyruvate to lactate, or a combination thereof. If the disease or disorder is characterized by cells exhibiting an Inverse Warburg effect metabolic phenotype, and the active agent can be one that shifts the metabolism of the cells from glucose oxidation to glycolysis, suppresses mitochondria-dependent apoptosis, decreases the oxidation of pyruvate, increases the conversion of pyruvate to lactate, or a combination thereof.

In some embodiments, the compositions and methods are utilized to select and treat a subject for a cancer, an inflammatory or autoimmune disease or disorder, a neurodegenerative disease or disorder, diabetes (e.g., type II diabetes), a neurological disorder, seizure disorder, cardiovascular disease, ischemia, or endometriosis.

In some embodiments, particularly those directed to treating cancer or an inflammatory or autoimmune disease or disorder, the active agent is a pyruvate dehydrogenase kinase inhibitor. In such embodiments, the criteria under step (b) for selecting a subject can include the diseased or disordered cells having a level of pyruvate dehydrogenase kinase that is increased relative to the control. In some embodiments, the dosage of pyruvate dehydrogenase kinase inhibitor administered to the subject is positively correlated with the level of pyruvate dehydrogenase kinase in the diseased or disordered cells. In preferred embodiments the pyruvate dehydrogenase kinase inhibitor is dichloroacetate (DCA), or an analogue, derivative, or conjugate thereof. In particularly preferred embodiments, the pyruvate dehydrogenase dichloroacetate, or an analogue, derivative, or conjugate thereof is a dichloroacetate (DCA) analogue targeted to the mitochondria. In some embodiments, when the diseased or disordered cells have a level of pyruvate dehydrogenase kinase that is not substantially increased, the same, or reduced relative to the control, the subject is administered an active agent the functions downstream of PDK, for example in the TCA, electron transport chain, during oxidative phosphorylation, etc.

Expression of HIF-1α and PDK expression are linked, and can be positively correlated. Thus in the foregoing methods, analysis of HIF-1α can be substituted and serve as proxy for PDK expression levels.

Methods of adjusting the dosage of DCA, or an analogue, derivative, or conjugate thereof, are also provided. For example, in some embodiments, although subjects may be selected for treatment, they may be given a lower dosage, less frequent administration, or the combination thereof (e.g., less than standard dosing regimen) of DCA, or an analogue, derivative, or conjugate thereof if the subject has at least one KGM allele, has at least one EGM allele, does not have at least one EGT allele, or a combination thereof at amino acid positions 32, 42, and 82 of the GSTz1/MAAI protein.

Suitable controls for the disclosed methods are known art, but generally can include, for example, a standard established by analysis of non-diseased cells from a single individual, or pooled or averaged values of like individuals, using the same assay as the test samples. In some embodiments the material for the control is non-diseased tissue from the subject. Typically, the control is derived from non-diseased cells of the same tissue or cell type as the cancer cells.

Any of the methods of selection and treatment can be coupled with a method of monitoring the efficacy of the active agent. The methods can include, for example, (a) comparing the level of a biomarker selected from the group consisting of hypoxia-inducible factor-1α (HIF-1α), lactate, FDG-PET, or a related glucose uptake measurement in a control biological sample, or scan in the case of FDG-PET, obtained from the subject before treatment begins to the level of the biomarker in one or more treatment biological samples (or scans) obtained after administration of the modulator begins, and (b) adjusting the dosage or frequency of administration of the modulator if the level of the biomarker is not altered relative to the control biological samples. In some embodiments, the assay is used to discontinue treatment. For example, in some embodiments, treatment is discontinued if the level of biomarker is not lower or higher than the control in a treatment biological sample obtained after at least 2, 3, 4, 5, or more administrations of the modulator. In particular embodiments, the biological samples are tissue samples such as tumor or biological fluid samples such as serum.

In some embodiments, when the active agent is dichloroacetate (DCA) or an analogue, derivative, or conjugate thereof, a method of selection and/or treatment can include (a) comparing the level the active agent, maleylacetoacetate, maleylacetone, or delta-aminolevulinate in a biological sample obtained from the subject after treatment begins, and (b) reducing the dosage or frequency of administration or discontinuing treatment with the active agent if the level the active agent, maleylacetoacetate, maleylacetone, or delta-aminolevulinate is above a threshold indicting hepatotoxicity or neurotoxicity. In preferred embodiments, the level of maleylacetone is compared in step (a). The biological sample can be, for example, urine.

In some embodiments, MPC level alone or in combination with HIF level is used to select a subject for treatment with an inhibitor of citrate transport or another transporter or enzyme related to formation or cycling of malate, citrate, or acetyl-CoA; or an inhibitor of glutaminolysis or a transporter or enzyme associated therewith such as glutaminase. In particular embodiments, the inhibitor targets the mitochondrial inner membrane citrate transport protein (CTP) (also referred to as tricarboxylate transport protein and SLC25A1). The method can include, for example, (a) detecting the level of MPC1, MPC2, or a combination thereof in diseased or disordered cells obtained from the subject; (b) selecting the subject for treatment if the level of MPC1, MPC2, or the combination thereof is reduced relative to a control; and optionally (c) administering the selected subjects an effective amount of an inhibitor of citrate transporter or another transporter or enzyme related to formation or cycling of malate, citrate, or acetyl-CoA; or an inhibitor of glutaminolysis or a transporter or enzyme associated therewith such as glutaminase, to treat the disease or disorder.

Another exemplary method can include, (a) detecting the level of HIF1, HIF2, or a combination thereof in diseased or disordered cells obtained from the subject that express wildtype, near wildtype, or not substantially reduced MPC1 or MPC2, or the combination thereof; (b) selecting the subject for treatment if the level of HIF1, HIF2, or the combination thereof is not reduced or is increased relative to a control; and optionally (c) administering the selected subjects an effective amount of an inhibitor of glutaminolysis or a transporter or enzyme associated therewith such as glutaminase, to treat the disease or disorder.

Some methods include (a) detecting the level of MPC1, MPC2, or a combination thereof in diseased or disordered cells obtained from the subject; (b) detecting the level of HIF1, HIF2, or a combination thereof in diseased or disordered cells obtained from the subject if the level of MPC1, MPC2, or the combination thereof is increased, the same, similar, or otherwise not substantially reduced relative to a control; (c) selecting the subject for treatment if the level of HIF1, HIF2, or the combination thereof is not reduced or is increased relative to a control; and optionally (d) administering the selected subjects an effective amount of an inhibitor of glutaminolysis or a transporter or enzyme associated therewith such as glutaminase, to treat the disease or disorder.

Exemplary inhibitors of citrate transport or another transporter or enzyme related to formation or cycling of malate, citrate, or acetyl-CoA; or glutaminolysis or a transporter or enzyme associated therewith such as glutaminase include, but are not limited to, 4-chloro-3-{[(3-nitrophenyl)amino]sulfonyl}benzoic acid, BMS 303141, MEDICA 16, SB 204990, BPTES, CB-839, 968, EGCG, AG-120, and AG-221.

### BRIEF DESCRIPTION OF THE DRAWINGS

FIGS. 1A and 1B are staining summaries derived from The Human Protein Atlas the level of antibody staining (i.e., protein expression) of MPC1 (FIG. 1A) and MPC2 (FIG. 1B) in various cancer tissues and comparing it with the protein expression levels in normal tissues.

FIG. 2 is a line graph showing the tumor inhibition relative to control following administration of 20 mpk of KULA2 to mice harboring HCT116 tumors and 18 mpk of KULA2 to mice harboring CT26 tumors.

FIG. 3 is a bar graph showing extracellular lactate in control or 6 hours after treatment with dichloroacetate (DCA), DCA after UK5099 pre-treatment, KULA2, and KULA2 after UK5099 pre-treatment. UK5099 pre-treatment DCA is dosed at 50 mM and KULA2 is dosed at 500 μM.

FIG. 4 is a line graph showing the change in cell viability (%) of MPC-positive A549 cells (*) and MPC-negative HCT116 cells (#) following treatment with increasing doses (μM) of a citrate transporter inhibitor.

FIG. 5 is a line graph showing the change in cell viability (%) of MPC-positive CT26 cells (*) and MPC-negative HCT116 cells (#) following treatment with increasing doses (μM) of a glutaminolysis inhibitor.

FIG. 6 is a line graph showing the change in cell viability (%) of HIF1-low, MPC-positive CT26 cells (*) and HIF1-high, MPC-positive A549 cells (#) following treatment with increasing doses (μM) of a glutaminolysis inhibitor.

### DETAILED DESCRIPTION OF THE INVENTION

### I. Definitions

As used herein, the term “biomarker” is anything that can be used as an indicator of a particular physiological state of an organism. For example a biomarker is the level(s) of a particular by-product, metabolite, mRNA or protein associated with the particular physiological state.

As used herein, the terms “optional” or “optionally” mean that the subsequently described event, circumstance, or material may or may not occur or be present, and that the description includes instances where the event, circumstance, or material occurs or is present and instances where it does not occur or is not present.

As used herein, the terms “subject,” “individual,” and “patient” refer to any individual who is the target of treatment using the disclosed compositions. The subject can be a vertebrate, for example, a mammal. Thus, the subject can be a human. The subjects can be symptomatic or asymptomatic. The term does not denote a particular age or sex.

As used herein, the term “treating” includes alleviating the symptoms associated with a specific disorder or condition and/or inhibiting the development or progression of the symptoms.

As used herein, the term “effective amount” or “therapeutically effective amount” means a dosage sufficient to treat, inhibit, or alleviate one or more symptoms of the disorder being treated or to otherwise provide a desired pharmacologic and/or physiologic effect. The precise dosage will vary according to a variety of factors such as subject-dependent variables (e.g., age, immune system health, etc.), the disease, and the treatment being effected.

As used herein, the term “increase” can refer to a level including the reference level or cut-off-value or to an overall increase of 5%, 10%, 20%, 25%, 30%, 40%, 50%, 60%, 70%, 80%, 85%, 90%, 95%, 96%, 97%, 98%, 99% or greater, in biomarker level detected by the methods described herein, as compared to the level of the same biomarker from a reference sample. In certain embodiments, the term increase refers to the increase in biomarker level, wherein the increased level is 0.1, 0.5, 1, 2, 3, 4, 5-fold or more than 5-fold higher compared to the level of the biomarker in a reference sample.

As used herein, the term “decrease” can refer to a level below the reference level or cut-off-value or to an overall reduction of 5%, 10%, 20%, 25%, 30%, 40%, 50%, 60%, 70%, 80%, 85%, 90%, 95%, 96%, 97%, 98%, 99% or greater, in biomarker level detected by the methods described herein, as compared to the level of the same biomarker from a reference sample. In certain embodiments, the term decrease refers to the decrease in biomarker level, wherein the decreased level is 0.1, 0.5, 1, 2, 3, 4, 5-fold or more than 5-fold lower compared to the level of the biomarker in a reference sample.

As used herein, the term “at a reference level” refers to a biomarker level that is the same as the level of the same biomarker, detected by the methods described herein, from a reference sample.

As used herein, the term “reference level” herein refers to a predetermined value. As the skilled artisan will appreciate the reference level is predetermined and set to meet the requirements in terms of e.g. specificity and/or sensitivity. These requirements can vary, e.g. from regulatory body to regulatory body. It may, for example, be that assay sensitivity or specificity, respectively, has to be set to certain limits, e.g. 80%, 90% or 95%. These requirements may also be defined in terms of positive or negative predictive values. Nonetheless, based on the disclosure herein, it is possible to arrive at the reference level meeting those requirements.

As used herein, the phrases “substantially similar” or “substantially the same,” denote a sufficiently high degree of similarity between two numeric values (for example, one associated with an antibody of the invention and the other associated with a reference/comparator antibody), such that one of skill in the art would consider the difference between the two values to be of little or no biological and/or statistical significance within the context of the biological characteristic measured by said values. The difference between said two values is, for example, less than about 50%, less than about 40%, less than about 30%, less than about 20%, and/or less than about 10% as a function of the reference/comparator value.

As used herein, the phrases “substantially reduced,” “substantially increased,” or “substantially different,” as used herein, denotes a sufficiently high degree of difference between two numeric values such that one of skill in the art would consider the difference between the two values to be of statistical significance within the context of the biological characteristic measured by said values. The difference between said two values is, for example, greater than about 10%, greater than about 20%, greater than about 30%, greater than about 40%, and/or greater than about 50% as a function of the value for the reference/comparator molecule.

### II. Methods of Selecting Subjects, Monitoring Efficacy, and Adjusting Dosage

Loss of mitochondrial pyruvate import has been reported as the cause of decreased mitochondrial pyruvate oxidation (Eboli et al., Biochim. Biophys. Acta, 460, 183-187, (1977); Paradies et al., Cancer Res., 43, 5068-5071 (1983)). Mitchondrial pryruvate carrier (MPC) is a multimeric complex that modulates pryruvate uptake (Bricker et al., Science, 337, 96-100 (2012); Herzig et al., Science, 337, 93-96 (2012)). The MPC contains two proteins, MPC1 and MPC2, the absence of either of which leads to a loss of mitochondrial pyruvate uptake and utilization in yeast, flies, and mammalian cells (Bricker et al., Science, 337,96-100 (2012); Herzig et al., Science, 337,93-96 (2012) (Colca et al., PLoS One, 8, e61551 (2013); Divakaruni et al., Proc. Natl. Acad. Sci. USA, 110,5422-5427 (2013); Li et al., Mol. Plant 7, 1508-1521 (2014); Patterson et al., J. Biol. Chem., 289,13335-13346 (2014); Rohatgi et al., PLoS One, 8, e62012 (2013); Timo´n-Go´mez et al., PLoS ONE, 8, e79405 (2013)). See also, Yang, et al., Molecular Cell, 56(3), 414-424 (2014), Vacanti, et al., Molecular Cell, 56(3):425-35 (2014), Sziosarek, et al., Molecular Cell, 56(3):343-344 (2014), Rampelt, et al., EMBO J., 34(7): 835-837 (2015), and Bender, et al., EMBO J., 34:911-924 (2015)).

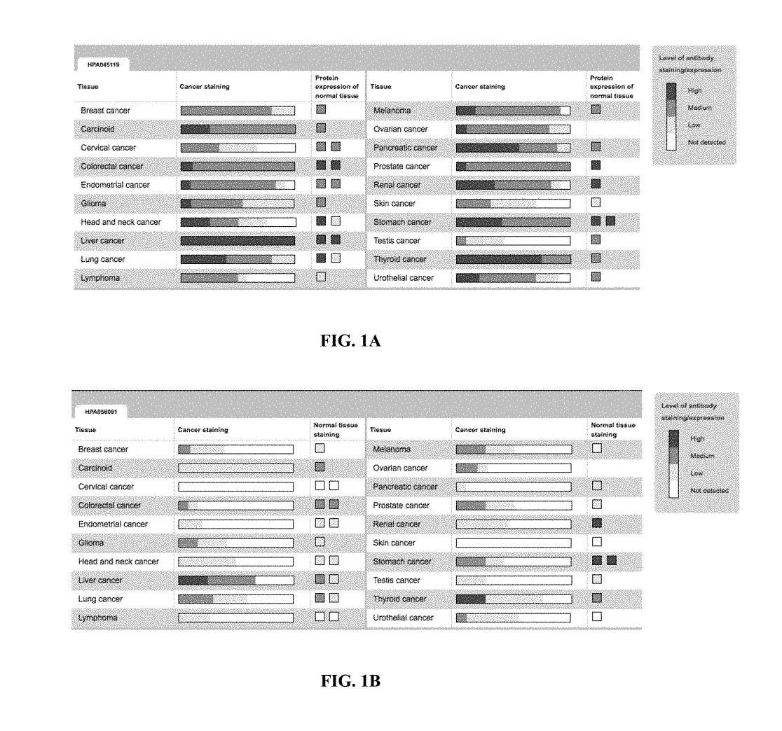

Furthermore, studies show that MPC expression or activity is lost in cancer (Schell, et al., Molecular Cell, 56:400-413 (2014)). Both genes, but particularly MPC1, are underexpressed or deleted in most cancers, and low expression correlates with poor survival. See also, FIGS. 1A and 1B. Experiments also show that when MPC expression is rescued, cells exhibited enhanced pyruvate oxidation and decreased glycolysis, consistent with reversal of the Warburg effect. While growth in standard adherent cell culture was unaffected, MPC re-expression impaired anchorage-independent growth, including in mouse xenograft assays, and was accompanied by decreased expression of stem cell markers. These data lead to a conclusion that decreased MPC expression promotes the Warburg effect and the maintenance of stemness in colon cancer cells (Schell, et al., Molecular Cell, 56:400-413 (2014)).

The experiments in the Example below show that the efficacy of pyruvate dehydrogenase kinase (PDK) inhibitors positively correlates with the level of MPC expression. The experiments show that therapies such a dichloroacetate and others that target downstream steps in pyruvate metabolism, the tricarboxylic acid (TCA) cycle, and oxidative phosphorylation can have little effect on metabolism if the cells have reduced or limited transport of the pyruvate into the mitochondria. Thus methods of determining the genotype, haplotype, or expression level of one or more biomarkers that contribute to pyruvate import into the mitochondria and other steps in pyruvate metabolism can be used to select subjects for treatment with agents that modulate pyruvate metabolism and steps downstream thereof including various points in the TCA cycle; citrate transport or another transporter or enzyme related to formation or cycling of malate, citrate, or acetyl-CoA; glutaminolysis or a transporter or enzyme associated therewith such as glutaminase; or oxidative phosphorylation.

A. Biomarkers for Selecting Subjects for Treatment

The methods of selecting subjects for treatment with an active agent that modifies pyruvate metabolism; the TCA cycle; citrate transport or another transporter or enzyme related to formation or cycling of malate, citrate, or acetyl-CoA; glutaminolysis or a transporter or enzyme associated therewith such as glutaminase; or oxidative phosphorylation can include detecting the level of a biomarker of pyruvate metabolism or transport, or the genotype or haplotype of the gene encoding the biomarker, in a sample obtain from the subject. Examples of biomarkers include, but are not limited to, mitochondrial pyruvate carrier (MPC), the pyruvate dehydrogenase complex (PDC), or the mitochondrial glutamine transporter. In some embodiments, the methods include assaying two, three, or all four biomarkers. Typically, subjects with samples that exhibit a substantially reduced level of biomarker compared to a control will not be selected for treatment, while subjects with substantially similar or an increased level of biomarker compared to a control can be selected for treatment.

1. Mitochondrial Pyruvate Carrier

The disclosed methods can include measuring the expression level of, or determining the genotype of the gene encoding, one or more MPCs in cells of the subject. As discussed above, expression of MPC1 and MPC2 can be reduced or absent in certain cells including cancer cells. Such cells are likely to have little or no response to pyruvate dehydrogenase kinase inhibitors such as dichloroacetate or other agents that modulate pyruvate metabolism, glutaminolysis or a transporter or enzyme associated therewith such as glutaminase; or dysfunction in other downstream steps in the TCA cycle and oxidative phosphorylation, or citrate transport or another transporter or enzyme related to formation or cycling of malate, citrate, or acetyl-CoA. Thus a method of selecting subjects for treatment with an active agent that modifies pyruvate metabolism; the TCA cycle; citrate transport or another transporter or enzyme related to formation or cycling of malate, citrate, or acetyl-CoA; glutaminolysis or a transporter or enzyme associated therewith such as glutaminase; or oxidative phosphorylation can include determining the expression level of one or more MPCs in cells isolated from the subject and selecting the subject for treatment, and optionally treating the subject with the active agent if the cells have at least 20%, 25%, 30%, 35%, 40%, 45%, 50%, 55%, 60%, 65%, 70%, 75%, 80%, 85%, 90%, 95%, 100%, or more than 100% of the expression of the MPC(s) relative to a control. In the most preferred embodiments, the subjects express one or both MPC proteins at a level (1) not substantially lower than, (2) equal to, or (3) greater than the average of wildtype.

In some embodiments, subjects are not selected for treatment if the cells do not have at least 20%, 25%, 30%, 35%, 40%, 45%, 50%, 55%, 60%, 65%, 70%, 75%, 80%, 85%, 90%, 95%, 100%, or more than 100% of the expression of the MPC(s) relative to a control. In some embodiments, although a subject may not be selected due to MPC expression, the subject can nonetheless be selected if mitochondrial glutamine transporter (discussed in more detail below) is high enough, for example, (1) not substantially lower than, (2) equal to, or (3) greater than the average of wildtype. This is because MPC and glutamine transporter can serve as two pathways to an active TCA cycle in the mitochondria. Thus, even if MPC expression is low, subjects may be selected for treatment with an agent that modifies pyruvate metabolism; the TCA cycle; citrate transport or another transporter or enzyme related to formation or cycling of malate, citrate, or acetyl-CoA; glutaminolysis or a transporter or enzyme associated therewith such as glutaminase; or oxidative phosphorylation if mitochondrial glutamine transporter is for example, 20%, 25%, 30%, 35%, 40%, 45%, 50%, 55%, 60%, 65%, 70%, 75%, 80%, 85%, 90%, 95%, 100%, or more than 100% of the expression of mitochondrial glutamine transporter relative to a control.

In some embodiments, a method of selecting subjects for treatment with an active agent that modifies pyruvate metabolism; the TCA cycle; citrate transport or another transporter or enzyme related to formation or cycling of malate, citrate, or acetyl-CoA; glutaminolysis or a transporter or enzyme associated therewith such as glutaminase; or oxidative phosphorylation can include determining the genotype of one or more alleles of one or more MPCs in cells of the subject. As discussed above, in some cancers the genes that encode MPC proteins are deleted or mutated. Thus, a method of selecting subjects for treatment with an active agent that modifies pyruvate metabolism; the TCA cycle; citrate transport or another transporter or enzyme related to formation or cycling of malate, citrate, or acetyl-CoA; glutaminolysis or a transporter or enzyme associated therewith such as glutaminase; or oxidative phosphorylation can include determining if one or more of the MPC genes in cells of the subject is deleted or mutated relative to a corresponding functional MPC gene and selecting the subject for treatment, and optionally treating the subject, with the active agent if the MPC gene in the cells is the same or similar to a functional MPC gene or otherwise believed to encode a functional MPC protein (e.g., mutations are synonymous mutations, or occur in non-coding regions such as introns, etc.). Subjects with function-improving mutations that increase expression of an MPC gene can also be selected for treatment.

a. Mitochondrial Pyruvate Carrier 1

Protein, mRNA, and gene sequences for mitochondrial pyruvate carrier 1 (MPC1) are known in the art.

For example, a protein sequence for MPC1 is

MAGALVRKAADYVRSKDFRDYLMSTHFWGPVANWGLPIAAINDMKKSPEI
ISGRMTFALCCYSLTFMRFAYKVQPRNWLLFACHATNEVAQLIQGGRLIK
HEMTKTASA

(:SEQ ID NO:1) (UniProtKB-Q9Y5U8 (MPC1_HUMAN), and Homo sapiens HSPC040 protein mRNA, complete cds).

An mRNA sequence (provided as cDNA) for MPC1 is

GTCGTGAGGCGGGCCTTCGGGCTGGCTCGCCGTCGGCTGCCGGGGGGTTG
GCCTGGGTGTCATTGGCTCTGGGAAGCGGCAGCAGAGGCAGGGACCACTC
GGGGTCTGGTGTCGGCACAGCCATGGCGGGCGCGTTGGTGCGGAAAGCGG
CGGACTATGTCCGAAGCAAGGATTTCCGGGACTACCTCATGAGTACGCAC
TTCTGGGGCCCAGTAGCCAACTGGGGTCTTCCCATTGCTGCCATCAATGA
TATGAAAAAGTCTCCAGAGATTATCAGTGGGCGGATGACATTTGCCCTCT
GTTGCTATTCTTTGACATTCATGAGATTTGCCTACAAGGTACAGCCTCGG
AACTGGCTTCTGTTTGCATGCCACGCAACAAATGAAGTAGCCCAGCTCAT
CCAGGGAGGGCGGCTTATCAAACACGAGATGACTAAAACGGCATCTGCAT
AACAATGGGAAAAGGAAGAACAAGGTCTTGAAGGGACAGCATTGCCAGCT
GCTGCTGAGTCACAGATTTCATTATAAATAGCCTCCCTAAGGAAAATACA
CTGAATGCTATTTTTACTAACCATTCTATTTTTATAGAAATAGCTGAGAG
TTTCTAAACCAACTCTCTGCTGCCTTACAAGTATTAAATATTTTACTTCT
TTCCATAAAGAGTAGCTCAAAATATGCAATTAATTTAATAATTTCTGATG
ATGTTTTATCTGCAGTAATATGTATATCATCTATTAGAATTTACTTAATG
AAAAACTGAAGAGAACAAAATTTGTAACCACTAGCACTTAAGTACTCCTG
ATTCTTAACATTGTCTTTAATGACCACAAGACAACCAACAGCTGGCCACG
TACTTAAAATTTTGTCCCCACTGTTTAAAAATGTTACCTGTGTATTTCCA
TGCAGTGTATATATTGAGATGCTGTAACTTAATGGCAATAAATGATTTAA
ATATTTGTTAAAAAAAAAAAAAAAAAAAAAAAAAAAAA

(SEQ ID NO:2) (Homo sapiens HSPC040 protein mRNA, complete cds).

A gene sequence for MPC1 can be found as part of Human DNA sequence from clone RP1-168L15 on chromosome 6q26-27, complete sequence GenBank: AL022069.1

b. Mitochondrial Pyruvate Carrier 2

Protein, mRNA, and gene sequences for mitochondrial pyruvate carrier 2 (MPC2) are known in the art.

For example, a protein sequence for MPC2 is

MSAAGARGLRATYHRLLDKVELMLPEKLRPLYNHPAGPRTVFFWAPIMK
WGLVCAGLADMARPAEKLSTAQSAVLMATGFIWSRYSLVIIPKNWSLFA
VNFFVGAAGASQLFRIWRYNQELKAKHK

(SEQ ID NO:3) (UniProtKB-O95563 (MPC2_HUMAN), and H. sapiens gene from PAC 295C6, similar to rat P044).

An mRNA sequence (provided as cDNA) for MPC2 is

CTCAGCGCCTCCGCCCCGGGGCCCCCGCTCACCCAGGTATCGACTCCGC
AGCCGGGACGGGTCCTCCAGCCCGAGGGACCTTTTCCTCACGTCCCACA
ACAGCCAGGGACGAGAACACAGCCACGCTCCCACCCGGCTGCCAACGAT
CCCTCGGCGGCGATGTCGGCCGCCGGTGCCCGAGGCCTGCGGGCCACCT
ACCACCGGCTCCTCGATAAAGTGGAGCTGATGCTGCCCGAGAAATTGAG
GCCGTTGTACAACCATCCAGCAGGTCCCAGAACAGTTTTCTTCTGGGCT
CCAATTATGAAATGGGGGTTGGTGTGTGCTGGATTGGCTGATATGGCCA
GACCTGCAGAAAAACTTAGCACAGCTCAATCTGCTGTTTTGATGGCTAC
AGGGTTTATTTGGTCAAGATACTCACTTGTAATTATTCCAAAAAATTGG
AGTCTGTTTGCTGTTAATTTCTTTGTGGGGGCAGCAGGAGCCTCTCAGC
TTTTTCGTATTTGGAGATATAACCAAGAACTAAAAGCTAAAGCACACAA
ATAAAAGAGTTCCTGATCACCTGAACAATCTAGATGTGGACAAAACCAT
TGGGACCTAGTTTATTATTTGGTTATTGATAAAGCAAAGCTAACTGTGT
GTTTAGAAGGCACTGTAACTGGTAGCTAGTTCTTGATTCAATAGAAAAA
TGCAGCAAACTTTTAATAACAGTCTCTCTACATGACTTAAGGAACTTAT
CTATGGATATTAGTAACATTTTTCTACCATTTGTCCGTAATAAACCATA
CTTGCTCGTATATA

(SEQ ID NO:4) (H. sapiens gene from PAC 295C6, similar to rat PO44).

A gene sequence for MPC2 can be found as part of Human DNA sequence from Human DNA sequence from clone RP1-295C6 on chromosome 1q24, complete sequence GenBank: Z97876.1.

2. Pyruvate Dehydrogenase Complex (PDC)

The pyruvate dehydrogenase complex facilitates conversion of pyruvate into acetyl-CoA by pyruvate decarboxylation (Swanson Conversion) thus linking the glycolysis metabolic pathway to the citric acid cycle. Pyruvate dehydrogenase is the first component enzyme of pyruvate dehydrogenase complex (PDC). PDC is a large complex containing many copies of each of three enzymes, pyruvate dehydrogenase (E1), dihydrolipoyl transacetylase (E2), and dihydrolipoyl dehydrogenase (E3). The inner core of the PDC is an icosahedral structure consisting of 60 copies of E2. At the periphery of the complex are: 30 copies of E1 (itself a tetramer with subunits α₂β₂) and 12 copies of E3 (a homodimer), plus 12 copies of an E3 binding protein that links E3 to E2.

Pyruvate dehydrogenase (E1) performs the first two reactions within the pyruvate dehydrogenase complex (PDC): a decarboxylation of pyruvate and a reductive acetylation of lipoic acid. Lipoic acid is then covalently bound to the second catalytic component enzyme of PDC, dihydrolipoamide acetyltransferase (E2). The reaction catalyzed by pyruvate dehydrogenase (E1) is considered to be the rate-limiting step for the pyruvate dehydrogenase complex (PDHc).

Inhibition of pyruvate dehydrogenase complex (PDC) activity contributes to the Warburg metabolic and malignant phenotype in cancer (McFate, et al.,J Biol Chem., 283(33): 22700-22708 (2008)). PDC inhibition occurs via enhanced expression of pyruvate dehydrogenase kinases 1-4 (PDK1-4), which results in inhibitory phosphorylation of the pyruvate dehydrogenase a (PDHα) subunit. Pyruvate dehydrogenase kinase inhibitors can reverse this phenomenon by blocking the phosphorylation of the PDC. However, if the cells have reduced expression of the PDC or any component thereof, such cells are likely to have little or no response to pyruvate dehydrogenase kinase inhibitors such as dichloroacetate or other agents that inhibit pyruvate metabolism, downstream steps in the TCA cycle, or oxidative phosphorylation. Likewise, activators downstream of the PDC that drive Inverse Warburg metabolism may have little or no effect if the activity of the PDC is impaired.

Thus a method of selecting subjects for treatment with an active agent that modifies pyruvate metabolism, the TCA cycle, or oxidative phosphorylation can include determining the expression level of the pyruvate dehydrogenase complex (PDC), or any component thereof such as pyruvate dehydrogenase or a subunit thereof, in cells isolated from the subject and selecting the subject for treatment, and optionally treating the subject with the active agent, if the cells have at least 20%, 25%, 30%, 35%, 40%, 45%, 50%, 55%, 60%, 65%, 70%, 75%, 80%, 85%, 90%, 95%, 100%, or more than 100% of the expression of the PDC, or any component thereof, relative to a control. In the most preferred embodiments, the subjects express one or more components of the PDC at a level (1) not substantially lower than, (2) equal to, or (3) greater than wildtype.

In some embodiments, a method of selecting subjects for treatment with an active agent that modifies pyruvate metabolism, the TCA cycle, or oxidative phosphorylation can include determining the genotype of one or more alleles of any one or more components of the PDC in cells of the subject. A method of selecting subjects for treatment with an active agent that modifies pyruvate metabolism, the TCA cycle, or oxidative phosphorylation can include determining if one or more of genes encoding a component the PDC in cells of the subject is deleted or mutated relative to a corresponding functional PDC gene and selecting the subject for treatment, and optionally treating the subject, with the agent if the PDC gene in the cells is the same or similar to a functional PDC gene or otherwise believed to encode a functional PDC protein (e.g., mutations are synonymous mutations, or occur in non-coding regions such as introns, etc.). Subjects with function-improving mutations that increase expression of a PDC gene can also be selected for treatment.

Components of the PDC are discussed above and include pyruvate dehydrogenase (E1), dihydrolipoyl transacetylase (E2), and dihydrolipoyl dehydrogenase (E3), and subunits thereof, for example, pyruvate dehydrogenase subunit α and subunit β. Sequences for human PDC component genes, mRNA, and proteins are known in the art. See, for example, the UniProt Accession Numbers provided in Table 1 below.

TABLE 1
UniProt Accession Numbers (“Entry”) for PDC Components

| | | | Gene | |
|---|---|---|---|---|
| Entry | Entry name | Protein names | names | Length |
| P08559 | ODPA_HUMAN | Pyruvate dehydrogenase E1 | PDHA1 PHE1A | 390 |
| | | component subunit alpha, | | |
| | | somatic form, mitochondrial (EC | | |
| | | 1.2.4.1) (PDHE1-A type I) | | |
| P11177 | ODPB_HUMAN | Pyruvate dehydrogenase E1 | PDHB PHE1B | 359 |
| | | component subunit beta, | | |
| | | mitochondrial (PDHE1-B) (EC | | |
| | | 1.2.4.1) | | |
| P10515 | ODP2_HUMAN | Dihydrolipoyllysine-residue | DLAT DLTA | 647 |
| | | acetyltransferase component of | | |
| | | pyruvate dehydrogenase | | |
| | | complex, mitochondrial (EC | | |
| | | 2.3.1.12) (70 kDa mitochondrial | | |
| | | autoantigen of primary biliary | | |
| | | cirrhosis) (PBC) | | |
| | | (Dihydrolipoamide | | |
| | | acetyltransferase component of | | |
| | | pyruvate dehydrogenase | | |
| | | complex) (M2 antigen complex | | |
| | | 70 kDa subunit) (Pyruvate | | |
| | | dehydrogenase complex | | |
| | | component E2) (PDC-E2) | | |
| | | (PDCE2) | | |
| P09622 | DLDH_HUMAN | Dihydrolipoyl dehydrogenase, | DLDGCSL, | 509 |
| | | mitochondrial (EC 1.8.1.4) | LAD, PHE3 | |
| | | (Dihydrolipoamide | | |
| | | dehydrogenase) (Glycine | | |
| | | cleavage system L protein) | | |

3. Glutamine Transporter

Glutamine enters the mitochondria through the mitochondrial glutamine transporter where through the glutaminolytic pathway it is converted into glutamate by glutaminase, and is reacted with pyruvate to form of α-ketoglutarate. Glutaminolysis serves as an important form of energy production cells with the Warburg phenotype, and high glutamine concentrations are associated with tumor progression, while reduced levels are connected to a differentiated, non-cancerous phenotype (Turowski, Cancer Res. 54 (22): 5974-5980 (1994), Spittler, et al., J. Nutr., 127 (11): 2151-2157 (1997)). During Warburg metabolism the a-ketoglutarate can serve as a substrate for a truncated form of the TCA terminating with malate and producing ATP. Furthermore, pyruvate depletion redirects glutamine metabolism to produce acetyl-CoA and citrate. Studies show that import of pyruvate into the mitochondria via MPC suppresses glutamate dehydrogenase (GDH) and glutamine-dependent acetyl-CoA formation, while inhibition of MPC activates GDH and reroutes glutamine metabolism to generate both oxaloacetate and acetyl-CoA, enabling persistent tricarboxylic acid (TCA) cycle function in the absence of glucose derived pyruvate (Yang, et al., Molecular Cell, 56(3):414-424 (2014)).

Thus, cells with little or no expression of glutamine transporter are likely to have little or no response to pyruvate dehydrogenase kinase inhibitors such as dichloroacetate or other agents that modulate pyruvate metabolism or other downstream steps in the TCA cycle and oxidative phosphorylation.

Thus a method of selecting subjects for treatment with an active agent that directly or indirectly modifies pyruvate metabolism, the TCA cycle, or oxidative phosphorylation can include determining the expression level of one or more glutamine transporters in cells isolated from the subject and selecting the subject for treatment, and optionally treating the subject with the active agent if the cells have at least 20%, 25%, 30%, 35%, 40%, 45%, 50%, 55%, 60%, 65%, 70%, 75%, 80%, 85%, 90%, 95%, 100%, or more than 100% of the expression of the glutamine transporter relative to a control. In the most preferred embodiments, the subjects express one or more glutamine transporter proteins at a level (1) not substantially lower than, (2) equal to, or (3) greater than wildtype.

In some embodiments, subjects are not selected for treatment if the cells do not have at least 20%, 25%, 30%, 35%, 40%, 45%, 50%, 55%, 60%, 65%, 70%, 75%, 80%, 85%, 90%, 95%, 100%, or more than 100% of the expression of the MPC(s) relative to a control. In some embodiments, although a subject may not be selected due to glutamine transporter expression, the subject can nonetheless be selected if MPC1 and/or MPC2 expression is high enough, for example, (1) not substantially lower than, (2) equal to, or (3) greater than the average of wildtype. As introduced above, this is because MPC and glutamine transport can serve as two pathways to an active TCA cycle in the mitochondria. Thus, even if mitochondrial glutamine transporter expression is low, subjects may be selected for treatment with an agent that modifies pyruvate metabolism, the TCA cycle, or oxidative phosphorylation if MPC1 and/or MPC2 is, for example, 20%, 25%, 30%, 35%, 40%, 45%, 50%, 55%, 60%, 65%, 70%, 75%, 80%, 85%, 90%, 95%, 100%, or more than 100% of the expression of MPC1 and/or MPC2 relative to a control.

In some embodiments, a method of selecting subjects for treatment with an active agent that directly or indirectly modifies pyruvate metabolism, the TCA cycle, or oxidative phosphorylation can include determining the genotype of one or more alleles of one or more glutamine transporters in cells of the subject. A method of selecting subjects for treatment with an active agent that directly or indirectly modifies pyruvate metabolism, the TCA cycle, or oxidative phosphorylation can include determining if one or more of the glutamine transporter genes in cells of the subject is deleted or mutated relative to a corresponding functional glutamine transporter gene and selecting the subject for treatment, and optionally treating the subject, with the active agent if the glutamine transporter gene in the cells is the same or similar to a functional glutamine transporter gene or otherwise believed to encode a functional glutamine transporter protein (e.g., mutations are synonymous mutations, or occur in non-coding regions such as introns, etc.). Subjects with function-improving mutations that increase expression of a glutamine transporter gene can also be selected for treatment.

The mammalian genomes encode a number of glutamine transporters, and including both plasma membrane and mitochondrial membrane forms. Typically, the biomarkers analyzed herein are mitochondrial membrane glutamine transporters (Pochini, et al., Frontiers in Chemistry, 2(61), 23 pages (2014) doi:10.3389/fchem.2014.00061, Sastrasinh and Sastrasinh, Am. J. Physiol., 257, F1050-F1058 (1989), Indiveri, et al., Biochem. J., 333(Pt 2), 285-290 (1998)).

B. Determining Pyruvate Dehydrogenase Kinase (PDK) Level

Pyruvate Dehydrogenase Kinase (PDK) is a kinase enzyme that inactivates pyruvate dehydrogenase by phosphorylating it using ATP. PDK thus reduces the ability of the pyruvate dehydrogenase complex to convert pyruvate to acetyl-coA, thereby facilitating an increase in the conversion of pyruvate to lactate in the cytosol. PDC inhibition in cancer cells is associated with normoxic stabilization of the malignancy-promoting transcription factor hypoxia-inducible factor-1α (HIF-1α) by glycolytic metabolites (McFate, et al., J Biol Chem., 283(33): 22700-22708 (2008)). As discussed in more detail below, in some embodiments, the pyruvate metabolism modulating agent administered to subjects for the treatment of cancer is a PDK inhibitor such as a dichloroacetate, which can reverse the metabolic effects of PDK. Likewise, PDK or activators thereof can induce the Inverse Warburg effect, thus increasing lactate metabolism and cellular longevity. Because subjects expressing increased levels of PDK can be good candidates for agents that modulate PDK, any of the disclosed methods can include measuring the expression level of, or determining the genotype of a gene encoding, PDK in the cells of a subject. Thus a method of selecting subjects for treatment with an active agent that modifies pyruvate metabolism, the TCA cycle, or oxidative phosphorylation can include determining the expression level of PDK in cells isolated from the subject and selecting the subject for treatment, and optionally treating the subject with an inhibitor of PDK, if the cells have at least an expression level of PDK that is equal to or greater than a control. In the most preferred embodiments, the subjects express one or both MPC proteins at a level (1) not substantially lower than, (2) equal to, or (3) greater than wildtype. In the most preferred embodiments, the expression level of PDK is at least 2, 3, 4, 5, or more-fold greater than the control.

In some embodiments, subjects with diseased cells having wildtype, lower than wildtype, or preferably substantially lower than wildtype levels of PDK can be selected for treatment with an active agent that acts downstream of PDK. Because PDK reduces the ability of the pyruvate dehydrogenase complex to convert pyruvate to acetyl-coA, cells that express wildtype or less than wildtype level of PDK can have at least some (and perhaps wildtype or greater than wildtype) levels of downstream metabolic activity (e.g., in the TCA, electron transport, OXPHOS, etc.). Thus even though the subject may not be a good candidate for a PDK inhibitor, the subject can nonetheless be selected for an agent that targets metabolic step downstream of pyruvate metabolism (e.g., in the TCA, electron transport etc.).

In some embodiments, a method of selecting subjects for treatment with an active agent that modifies pyruvate metabolism, the TCA cycle, or oxidative phosphorylation can include determining the genotype of one or more alleles of a PDK in cells of the subject. Thus, a method of selecting subjects for treatment with an active agent that modifies pyruvate metabolism, the TCA cycle, or oxidative phosphorylation can include determining if PDK in cells of the subject is mutated relative to a corresponding functional PDK gene and selecting the subject for treatment, and optionally treating the subject, with an inhibitor of PDK, if the PDK gene in the cells is found or believed to encode a PDK with increased expression or function (e.g., mutations in regulatory sequences that increase expression, function-gaining mutations that increase enzyme function such substrate recognition or turnover, etc.) relative to wildtype. In some embodiments, subjects are selected even if the gene is not mutated or includes a loss of function mutation relative to wildtype, however, generally, expression of PDK is at least wildtype or preferably greater than wildtype in the cells for a subject to be selected for treatment with a PDK modulator such as the PDK inhibitor dichloroacetate.

In some embodiments, the level of PDK is also taken into consideration when determining dosage. For example, a subject with a relative higher level of PDK expression may be given a larger dose and/or more frequent treatment than a subject with a relatively lower level of PDK. Thus the dosage and/or frequency of administration of a PDK inhibitor such as dichloroacetate or an analogue, derivative, or conjugate thereof to a subject can be positively correlated with PDK levels in the subject.

Sequences for human PDK genes, mRNA, and proteins are known in the art. See, for example, the UniProt Accession Numbers UniProtKB: Q15118 (PDK1_HUMAN) for human PDK1, Q15119 (PDK2_HUMAN) for human PDK2, Q15120 (PDK3_HUMAN) for human PDK3, and Q16654 (PDK4_HUMAN) for human PDK4.

Expression of HIF-1α and PDK expression are linked, and can be positively correlated. Thus HIF-1α can serve as proxy for PDK expression levels. It will also be appreciated that HIF-1α can be substituted for PDK in both the subject selection and treatment selection methods discussed above. Use of HIF-1α as biomarker for determining treatment efficacy and adjusting dosage, including preferred methods for measuring HIF-1α in a biological sample, is discussed in more detail below.

C. Analysis of DCA Glutathione Transferase (GSTZ1) Alleles

Additionally, any of the disclosed methods can include determining the genotype or haplotype of the gene encoding glutathione transferase (GSTZ1) in a subject. The result of the GSTz1/MAAI genotyping or haplotyping can be used to adjust dosage of dichloroacetate and analogues, derivatives, and conjugates thereof before or during treatment. The GSTz1/MAAI genotype or haplotype can be determined in a subject at any time before or during treatment.

Dichloroacetate is dehalogenated to glyoxylate by the zeta-1 family isoform of glutathione transferase (GSTz1). This enzyme is identical to maleylacetoacetate isomerase (MAAI), the penultimate enzyme of the phenylalanine/tyrosine catabolic pathway. Polymorphisms in the GSTz1/MAAI gene (GSTZ1 SNPs: rs7975 (g.5696G>A, Glu32Lys, E32K), rs7972 (g.5726G>A, Gly42Arg, G42R), and rs1046428 (g.6772C>T, Thr82Met, T82M) modify the kinetics of DCA and, consequently, the risk of adverse effects from the drug. GSTz1/MAAI haplotype clearly segregated subjects into fast and slow DCA metabolizers. Those who metabolized DCA slowly showed markedly delayed plasma clearance, increased excretion of unmetaboized drug and increased urinary accumulation of potentially toxic tyrosine metabolites. Therefore, the GSTz1/MAAI haplotype can predict the toxicogenetics of DCA and analogues, derivatives, and conjugates thereof, and this information can be used prospectively to adjust drug dosing and mitigate risk of adverse events when using the drugs.

Sequences for human GSTz1/MAAI genes, mRNA, and proteins are known in the art. See, for example, the UniProt Accession Number UniProtKB-O43708 (MAAI_HUMAN), which provides the canonical sequence below. This other full length GSTz1/MAAI sequences can as a reference sequence for protein for amino acid positions 32, 42, and 82 of GSTz1/MAAI the haplotypes discussed above.

MQAGKPILYSYFRSSCSWRVRIALALKGIDYKTVPINLIKDRGQQFSKD
FQALNPMKQVPTLKIDGITIHQSLAIIEYLEEMRPTPRLLPQDPKKRAS
VRMISDLIAGGIQPLQNLSVLKQVGEEMQLTWAQNAITCGFNALEQILQ
STAGIYCVGDEVTMADLCLVPQVANAERFKVDLTPYPTISSINKRLLVL
EAFQVSHPCRQPDTPTELRA

(SEQ ID NO:5). For purposes of illustration, the haplotype of SED ID NO:5 (UniProtKB-O43708 (MAAI_HUMAN)), is KRM at amino acid positions 32, 42, and 82 of SEQ ID NO:5. The positions are bolded and underlined in SEQ ID NO:5. Table 2, which provides natural variants with respect to SEQ ID NO:5, is adapted from information available through UniProtKB-O43708 (MAAI_HUMAN).

TABLE 2
Natural Variants of SEQ ID NO: 5

| Feature | | | Feature |
|---|---|---|---|
| key | Position(s) | Description | identifier |
| Natural | 32-32 | K → E in allele GSTZ1*C. | VAR_009705 |
| variant | | Corresponds to variant rs7975 | |
| Natural | 42-42 | R → G in allele GSTZ1*B and | VAR_009706 |
| variant | | allele GSTZ1*C. Corresponds to | |
| | | variant rs7972 | |
| Natural | 82-82 | M → T.7. Corresponds to variant | VAR_009707 |
| variant | | rs1046428 | |
| Natural | 133-133 | N → H.1. Corresponds to variant | VAR_014505 |
| variant | | rs2234955 | |

Methods of haplotyping GSTz1/MAAI and adjusting DCA drug dosing accordingly are described in U.S. Patent Application No. 2013/0090382. In some embodiments, the GSTz1/MAAI haplotype can include one or two of the following: a KGM allele, a EGM allele, a EGT allele, a KGT allele, and a KRT allele with reference to amino acid positions 32, 42, and 82 of the GSTz1/MAAI protein as introduced above. Subjects that possess at least one KGM or EGM allele may be at a particularly heightened risk for developing adverse drug effects, unless dose adjustments are made. Moreover, GSTz1/MAAI genotype may confer added risk to populations who are chronically exposed to environmental levels of DCA or its precursors and/or to chronic consumption of protein-enriched diets. Thus in some embodiments, a subject may be administered a less than standard dosing regimen of DCA or analogue, derivative, or conjugate there if the subject: (1) has at least one KGM allele, (2) has at least one EGM allele, (3) does not have at least one EGT allele, or a combination thereof at amino acid positions 32, 42, and 82 of the GSTz1/MAAI protein. In some embodiments, a subject may be administered a less than standard dosing regimen of DCA or analogue, derivative, or conjugate there if the subject has KRT allele homozygosity.

D. Methods of Measuring Efficacy and Adjusting Dosage

Methods of determining the efficacy and optionally adjusting the dosage of an active agent that modifies pyruvate metabolism; the TCA cycle; citrate transport or another transporter or enzyme related to formation or cycling of malate, citrate, or acetyl-CoA; glutaminolysis or a transporter or enzyme associated therewith such as glutaminase;, or oxidative phosphorylation are also provided. The methods can be used alone or coupled with any of the other methods disclosed herein.

1. HIF-1α, Lactate, Fluorodeoxyglucose (18F)

The methods can include measuring levels of hypoxia-inducible factor-1α (HIF-1α), lactate, fluorodeoxyglucose (also referred to as fluorodeoxyglucose 18F, fludeoxyglucose F18, and abbreviated [18F]FDG, 18F-FDG, FDG FDG-PET) or a related agent for measuring glucose uptake in a patient or from a biological sample from a subject to determine treatment efficacy and, if needed, adjust dosage or frequency of administration during treatment.

As introduced above, pyruvate dehydrogenase kinase (PDK)-driven PDC inhibition in cancer cells is associated with stabilization of the malignancy-promoting transcription factor hypoxia-inducible factor-1α (HIF-1α) by glycolytic metabolites (McFate, et al., J Biol Chem., 283(33): 22700-22708 (2008), Velpula, et al., Cancer Res., 15;73(24):7277-89 (2013)). Studies also show that HIF-1α and vascular endothelial growth factor (VEGF) are serum tumor markers, the levels of which can be effected by treatment in some cancer types (Liang, et al., Asian Pac J Cancer Prev., 14(6):3851-4 (2013)). Because expression of HIF-1α and other cytokines can be modulated through the activity of PDK, serum levels of HIF-1α can serve a proxy for efficacy of therapeutic interventions that target pyruvate metabolism, the TCA cycle, or oxidative phosphorylation. Sequences for human HIF-1α genes, mRNA, and proteins are known in the art. See, for example, the UniProt Accession Number UniProtKB-Q16665 (HIF1A_HUMAN).

Increased glucose uptake and accumulation of lactate are common features of cancer cells. Conversely, cells under Inverse Warburg metabolism may exhibit relatively reduced glucose uptake and accumulation of lactate. Thus, similar to HIF-1α, lactate, fluorodeoxyglucose (18F) and other agents for measuring glucose uptake can be used as proxies for efficacy of therapeutic interventions that target pyruvate metabolism, the TCA cycle, or oxidative phosphorylation. Applications of fluorodeoxyglucose (18F) in cancer treatment and including methods of using FDG-PET as a measure of treatment efficacy are reviewed in Kelloff, et al., Clin Cancer Res., 11: 2785-2808 (2005).

HIF-1α and lactate levels are most typically measured in an extracellular sample from the subject. In preferred embodiments, the sample is a fluid sample such as serum. The sample can also be aspirate (e.g., cell-free aspirate) from a tissue or tumor microenvironment. In other embodiments, intracellular levels, extracellular levels or a combination thereof are analyzed in a tissue or cellular sample (e.g., by immunohistochemistry, etc.).

Fluorodeoxyglucose (18F) is 2-deoxy-2-(18F)fluoro-D-glucose, a glucose analog, with the positron-emitting radionuclide fluorine-18 substituted for the normal hydroxyl group at the 2′ position in the glucose molecule. Uptake of fluorodeoxyglucose (18F) by tissues is an indicator for the uptake of glucose that can be visualized by positron emission tomography (PET) imaging. Thus, the disclosed methods typically involve measuring intracellular or tissue associated fluorodeoxyglucose (18F), and can be carried out non-invasively using PET imaging. Thus, in preferred embodiments, the biological sample in which fluorodeoxyglucose (18F) is measured is not an isolated sample, but rather the entire subject.

For example, the level of HIF-1α or lactate can be measured in a biological sample (e.g., an extracellular biological sample such as serum) from the subject before treatment with an agent that inhibits pyruvate metabolism, the TCA cycle, or oxidative phosphorylation and again one or more times after treatment. Fluorodeoxyglucose (18F) or another agent for measuring glucose uptake can be administered to the subject and measured in cells of the subject before treatment with an agent that inhibits pyruvate metabolism, the TCA cycle, or oxidative phosphorylation and again one or more times after treatment. A reduction in the level of HIF-1α or lactate, or a reduction in intracellular or tissue associated (cellular uptake) fluorodeoxyglucose (18F) or other agent for measuring glucose uptake in the sample can indicate that the treatment is effective. Preferably the treatment reduces the level of HIF-1α or lactate (e.g., in an extracellular biological sample such as serum), or cellular uptake of fluorodeoxyglucose (18F) or other agent by at least 20%, 25%, 30%, 35%, 40%, 45%, 50%, 55%, 60%, 65%, 70%, 75%, 80%, 85%, 90%, 95%, or 100% relative to the level prior to treatment. In some embodiments, if the level of HIF-1α or lactate, or cellular uptake of fluorodeoxyglucose (18F) or other agent is not reduced after, for example, 1, 2, 3, 4, 5, or more treatments, by for example, at least 20%, 25%, 30%, 35%, 40%, 45%, 50%, 55%, 60%, 65%, 70%, 75%, 80%, 85%, 90%, 95%, or 100%, the dosage of the treatment, frequency of administration of the treatment, or both is increased. In other embodiments, treatment may be discontinued.

Similarly, the level of HIF-1α or lactate can be measured in an biological sample from the subject (e.g., an extracellular biological sample such as serum) before treatment with an agent that increases pyruvate metabolism, the TCA cycle, or oxidative phosphorylation and again one or more times after treatment. In some embodiments, fluorodeoxyglucose (18F) or another agent for measuring glucose uptake is administered to the subject and measured in cells of the subject before treatment with an agent that increases pyruvate metabolism, the TCA cycle, or oxidative phosphorylation and again one or more times after treatment. An increase in the level of HIF-1α or lactate, or an increase in intracellular or tissue associated (cellular uptake) fluorodeoxyglucose (18F) or other agent for measuring glucose uptake in the sample can indicate that the treatment is effective. Preferably the treatment increases the extracellular (e.g., in an extracellular biological sample such as serum) level of HIF-1α or lactate, or cellular uptake of fluorodeoxyglucose (18F) or other agent by at least 20%, 25%, 30%, 35%, 40%, 45%, 50%, 55%, 60%, 65%, 70%, 75%, 80%, 85%, 90%, 95%, or 100% relative to the level prior to treatment. In some embodiments, if the level of HIF-1α or lactate, or cellular uptake of fluorodeoxyglucose (18F) or other agent is not increased after, for example, 1, 2, 3, 4, 5, or more treatments, by for example, at least 20%, 25%, 30%, 35%, 40%, 45%, 50%, 55%, 60%, 65%, 70%, 75%, 80%, 85%, 90%, 95%, or 100%, the dosage of the treatment, frequency of administration of the treatment, or both is increased. In other embodiments, treatment may be discontinued.

2. Dichloroacetate-Specific Biomarkers

GSTz1/MAAI inhibition by DCA results in the accumulation of the potentially hepatotoxic tyrosine intermediates maleylacetoacetate and maleylacetone, and of delta-aminolevulinate, a precursor of heme synthesis that has been associated with neurotoxic effects, including peripheral neuropathy (U.S. Published Application No. 2013/0090382 and references cited therein). Reversible increases in serum transaminases and reversible peripheral neuropathy have been reported in association with chronic DCA exposure. As discussed above, DCA clearance is linked to the subject's GSTz1/MAAI haplotype, and there is a strong association between plasma clearance of DCA and the urinary concentration of both DCA and maleylacetone. For example, subjects that were KRT homozygotes, or KGM or EGM heterozygotes exhibited reduced DCA kinetics, and those that lacked a EGT wildtype allele had the highest urinary concentration of maleylacetone (U.S. Published Application No. 2013/0090382).

Thus, for subjects being administered a DCA compound such as DCA or an analogue, derivative, or conjugate thereof, one or more biomarkers selected from the DCA compound itself, maleylacetoacetate, maleylacetone, and delta-aminolevulinate can be monitored in a biological sample from the subject one or more times over the course of the treatment. If the level of the DCA compound, maleylacetoacetate, maleylacetone, or delta-aminolevulinate exceeds a threshold level, the dosage or frequency of administration of DCA or an analogue, derivative, or conjugate thereof can be reduced or terminated. In preferred embodiments, the biomarker is maleylacetone.

Typically the threshold level is set below a level that will cause substantial toxicity to the subject, for example hepatotoxic or neurotoxic effects. Threshold levels can be determined experimentally or adopted from art recognized levels such as those described in U.S. Published Application No. 2013/0090382 and references cited therein.

In some embodiments, biological sample is blood, serum, or most preferably urine.

### III. Methods of Detecting Biomarkers

The methods disclosed herein can include detecting levels of expression of a biomarker, in a subject or a biological sample obtained from the subject, and comparing them to a control. Detecting alterations in the expression level of a biomarker can include measuring the level of protein or mRNA of the biomarker and comparing it to a control. Additionally, or alternatively, the methods can include genotyping or haplotyping the gene encoding the biomarker in a subject or a biological sample obtained from the subject, and comparing it to a control. In some embodiments, the biological sample is one that is isolated from the subject. In some embodiments, such as those in which in vivo imaging is employed, the subject serves are the biological sample.

A. Biological Samples

A biological sample can be obtained from an individual for use in the methods and bioassays disclosed herein. In some embodiments, the sample is a tissue biopsy or cells obtained from the subject. As discussed in more detail above, many of the assays can involve determining the expression level, genotype, or haplotype of a biomarker in a tumor sample from a subject.

As discussed in more detail below, the subject can be one with a disease or disorder in need of treatment. The biological sample can come for cells that characteristic of or otherwise effected by the disease or disorder. The biological sample can include a single cell, or preferable includes multiple cells. The biological sample can be tissue. Thus, in preferred embodiments, the biological sample is obtained from a tissue or organ that will exhibit symptoms or is otherwise associated with disease or disorder to be treated.

For example, if the subject if the subject has cancer, the cells of the biological sample are typically cancer cells. In preferred embodiments, the biological sample includes cancer cells obtained from a tumor. In some embodiments, the biological sample includes cancer cells that are not obtained from a tumor. For example, in some embodiments, the cancer cells are circulating cancer cells. The biological sample can include other components or cells that are not cancer cells. For example, the sample can include non-cancerous cells, tissue, etc. In preferred embodiments, the biological sample includes cancer cells isolated or separated away from normal tissue. In some embodiments, the biological sample is obtained from a cancerous tissue or organ. It will be appreciated that the above embodiments directed to cancer are exemplary, and can be analogously applied to biological samples for other diseases and disorders including those discussed herein.

A biological sample can be obtained from the subject using a variety of methods that are known in the art. In some embodiments, the sample is a tissue biopsy, for example a punch biopsy. The sample should be handled in accordance with the method of detection that will be employed.

In some embodiments, a biological sample that is of tissue or cellular origin can be solubilized in a lysis buffer optionally containing a chaotropic agent, detergent, reducing agent, buffer, and salts. The conditions for handling biological samples that are analyzed for mRNA level may be different than the conditions for handling biological samples that are analyzed for protein level, and such conditions are known in the art. If the sample is a blood sample that includes clotting factors (e.g., a whole blood sample), the preparation may include an anti-coagulant.

In some embodiments, for example, measuring the level of extracellular HIF-1α or lactate, the biological sample can be a biological fluid sample taken from a subject. Examples of biological samples include urine, barbotage, blood, serum, plasma, tears, saliva, cerebrospinal fluid, tissue, lymph, synovial fluid, or sputum etc. A biological fluid sample can be whole blood, or more preferably serum or plasma. Serum is the component of whole blood that is neither a blood cell (serum does not contain white or red blood cells) nor a clotting factor. It is the blood plasma with the fibrinogens removed. Accordingly, serum includes all proteins not used in blood clotting (coagulation) and all the electrolytes, antibodies, antigens, hormones, and any exogenous substances (e.g., drugs and microorganisms). The sample can be diluted with a suitable diluent before the sample is analyzed.

B. Methods of Detecting Expression Levels

The detection of mRNA, polypeptides and proteins in a biological sample obtained from a subject is made possible by a number of conventional methods that are known in the art. The methods can be cell-based or cell-free assays.

For example, mRNA levels can be determined using assays, including, but not limited to, RT-PCR, reverse transcription real-time PCR (RT-qPCR), transcriptome analysis using next-generation sequencing, array analysis, digital PCR, and northern analysis. In a preferred embodiment, the method includes detecting the level of a biomarker in mRNA isolated from cells of the subject. In some embodiments, a probe for detecting a biomarker is designed to hybridize with the nucleic acid sequence encoding the biomarker, or a compliment thereof.

Protein expression can be detected using routine methods, such as immunodetection methods, mass spectroscopy, or high performance liquid chromatography (HPLC). In a preferred embodiment, the method includes detecting the level of biomarker protein or polypeptide, or a combination thereof in protein isolated from cells of the subject.

Some methods include an immunoassay whereby polypeptides of the biomarker are detected by their interaction with a biomarker-specific antibody. The biomarker can be detected in either a qualitative or quantitative manner. Exemplary immunoassays that can be used for the detection of biomarker polypeptides and proteins include, but are not limited to, radioimmunoassays, ELISAs, immunoprecipitation assays, Western blot, fluorescent immunoassays, and immunohistochemistry, flow cytometry, protein arrays, multiplexed bead arrays, magnetic capture, in vivo imaging, fluorescence resonance energy transfer (FRET), and fluorescence recovery/localization after photobleaching (FRAP/FLAP).

It will be appreciated that some immunoassays, for example ELISAs, can require two different biomarker specific antibodies or ligands (e.g., a capture ligand or antibody, and a detection ligand or antibody). In certain embodiments, the protein biomarker is captured with a ligand or antibody on a surface and the protein biomarker is labeled with an enzyme. In one example, a detection antibody conjugated to biotin or streptavidin—to create a biotin-streptavidin linkage to an enzyme that contains biotin or streptavidin. A signal is generated by the conversion of the enzyme substrate into a colored molecule and the intensity of the color of the solution is quantified by measuring the absorbance with a light sensor. Contemplated assays may utilize chromogenic reporters and substrates that produce an observable color change to indicate the presence of the protein biomarker. Fluorogenic, electrochemiluminescent, and real-time PCR reporters are also contemplated to create quantifiable signals.

Some assays optionally including fixing one or more antibodies to a solid support to facilitate washing and subsequent isolation of the complex, prior to contacting the antibody with a sample. Examples of solid supports include glass or plastic in the form of, e.g., a microtiter plate, a stick, a bead, or a microbead. Antibodies can also be attached to a probe, substrate or a ProteinChip® array.

Flow cytometry is a laser based technique that may be employed in counting, sorting, and detecting protein biomarkers by suspending particles in a stream of fluid and passing them by an electronic detection apparatus. A flow cytometer has the ability to discriminate different particles on the basis of color. Differential dyeing of particles with different dyes, emitting in two or more different wavelengths allows the particle to be distinguished. Multiplexed analysis, such as FLOWMETRIX™ is discussed in Fulton, et al., Clinical Chemistry, 43(9):1749-1756 (1997) and can allow one to perform multiple discrete assays in a single tube with the same sample at the same time.

In some specific embodiments, the biomarker level(s) are measured using Luminex xMAP® technology. Luminex xMAP® is frequently compared to the traditional ELISA technique, which is limited by its ability to measure only a single analyte. The differences between ELISA and Luminex xMAP® technology center mainly on the capture antibody support. Unlike with traditional ELISA, Luminex xMAP® capture antibodies are covalently attached to a bead surface, effectively allowing for a greater surface area as well as a matrix or free solution/liquid environment to react with the analytes. The suspended beads allow for assay flexibility in a singleplex or multiplex format.

Commercially available formats that include Luminex xMAP® technology includes, for example, BIO-PLEX® multiplex immunoassay system which permits the multiplexing of up to 100 different assays within a single sample. This technique involves 100 distinctly colored bead sets created by the use of two fluorescent dyes at distinct ratios. These beads can be further conjugated with a reagent specific to a particular bioassay. The reagents may include antigens, antibodies, oligonucleotides, enzyme substrates, or receptors. The technology enables multiplex immunoassays in which one antibody to a specific analyte is attached to a set of beads with the same color, and the second antibody to the analyte is attached to a fluorescent reporter dye label. The use of different colored beads enables the simultaneous multiplex detection of many other analytes in the same sample. A dual detection flow cytometer can be used to sort out the different assays by bead colors in one channel and determine the analyte concentration by measuring the reporter dye fluorescence in another channel.

In some specific embodiments, the biomarker(s) levels are measured using Quanterix's SIMOA™ technology. SIMOA™ technology (named for single molecule array) is based upon the isolation of individual immunocomplexes on paramagnetic beads using standard ELISA reagents. The main difference between Simoa and conventional immunoassays lies in the ability to trap single molecules in femtoliter-sized wells, allowing for a “digital” readout of each individual bead to determine if it is bound to the target analyte or not. The digital nature of the technique allows an average of 1000× sensitivity increase over conventional assays with CVs <10%. Commercially available SIMOA™ technology platforms offer multiplexing options up to a 10-plex on a variety of analyte panels, and assays can be automated.

Multiplexing experiments can generate large amounts of data. Therefore, in some embodiments, a computer system is utilized to automate and control data collection settings, organization, and interpretation.

C. Genotyping and Haplotyping

Method of genotyping and haplotyping subjects for genes encoding biomarkers are known in the art and can include determining the entire sequence of the biomarker gene or a subsequence thereof in coding or non-coding regions. The methods can include, or be limited to, determining the sequence at one or more single nucleotide polymorphisms (SNPs). Methods of sequencing and genotyping genes and SNPs are known in the art and can include, for example, polymerase chain reaction (PCR), DNA sequencing, allele specific oligonucleotide (ASO) probes, hybridization to DNA or SNP microarrays or beads, dynamic allele-specific hybridization (DASH), molecular beacons, restriction fragment length polymorphism (RFLP), flap endonuclease (FEN)-based methods, primer extension, Taq DNA polymerase's 5′-nuclease activity-based assays (e.g., TaqMan), oligonucleotide ligation (detected by, for example, detected by gel electrophoresis, MALDI-TOF mass spectrometry or by capillary electrophoresis), single strand conformation polymorphism, temperature gradient gel electrophoresis, denaturing high performance liquid chromatography (DHPLC), High Resolution Melting analysis, DNA mismatch-binding proteins, etc.

Typical protocols for evaluating the status of genes and gene products are found, for example in Ausubel et al. (eds.), Current Protocols In Molecular Biology (1995), Units 2 (Northern Blotting), 4 (Southern Blotting), 15 (Immunoblotting) and 18 (PCR Analysis).

D. Controls

The methods disclosed herein typically including comparing the level of the biomarker detected in a sample obtained from the subject to a control. Suitable control will be known to one of skill in the art. Controls can include, for example, standards obtained from healthy subjects, such as subjects without the disease or disorder, or non-diseased tissue from the same subject. A control can be a single or more preferably pooled or averaged values of like individuals using the same assay. Reference indices can be established by using subjects that have been diagnosed with the disease or disorder with different known disease severities or prognoses. The control biological sample(s) can be assayed using the same methods as the test sample.

### IV. Devices and Kits for Detection of Biomarkers

Devices and kits for detection of biomarkers are also disclosed. Using the methods and systems of the present disclosure, several types of markers can be detected. The marker or markers being detected may indicate whether a subject has a cancer treatable with an active agent that modifies pyruvate metabolism, the TCA cycle, or oxidative phosphorylation, selecting the subject for treatment, determining the efficacy of the treatment, and adjusting the treatment dosage and frequency. The marker being detected may be a nucleic acid (or polynucleotide), protein, peptide, etc. The marker being detected can determine the format of the test (i.e., assay, strip, etc.), and/or the type of biomolecular recognition element (BRE) (e.g. antibodies, antigens, etc.) being used to detect the marker. The marker being detected may be a single marker or a combination of markers. The marker being detected may be specific to one condition or multiple conditions.

There may be provided a test or support surface used for performing a test for detecting the presence of a selected marker(s). The test or support surface may be coated with/hold the selected detection antibodies, etc. specific to the marker(s) being detected.

The device or kit typically includes reagents and/or apparatus that can be used to carry out the test. Some kits include an apparatus that includes a support surface for the detection of the marker. The surface, can be, for example a surface on which the selected detection antibodies, etc. can be coated/held for detection of the selected marker(s). In some embodiments, the test or support surface may be part of an assay having one or more containers (or wells). The test or support surface may be the inner surface of a well or container. The inner surface of one or more wells or containers may be coated with the detection antibody specific to the marker(s) being detected.

Any appropriate assay or ELISA (sandwich, indirect, competitive, reverse, etc.) can be provided as part of the kit or device. For example, the kits or device can provide a polystyrene microplate, having wells/containers with inner surfaces capable of being coated with antibody. These inner surfaces may or may not be treated with substances known in the art to promote or enhance coating. For example the surface can be a maxisorp, POLYSORP, medisorp, MINISORP or COVALINK surface. Each well or container may be white or opaque to allow for easier visualization of any color, or any visually detectable change, occurring in or on the well or container. It will be appreciated that the size, surface area, total and/or working volumes, appearance, and/or color/visual parameters and/or qualities can be modified as desired within the scope of the present disclosure.

In some embodiments, the test or support surface may be part of a vial (or container or well), a test strip, a chromatography substrate, a gene chip, a SNAP test, or any other diagnostic test or test system used for detecting markers. The test or support surface may be made of paper, plastic, glass, metal, etc. and take several forms such as paddle, beads, wells, electrodes, etc.

The kit or device can include an appropriate biomolecular recognition element (BRE), for detection of the biomarker. In some embodiments, the test surface is coated with the BRE (e.g., the detection antibody). In some embodiments, non-specific adsorption to the test surfaces coated with a BRE (e.g. the detection antibody), such as the coated well/container of an assay, may be minimized by blocking the test surface with a blocking agent. The blocking agent may be one or more proteins, sugars and/or polymers such as bovine serum albumin, gelatin, polyethylene glycol, sucrose, etc.

The coated surface, such as the coated well/container of an assay, may be coated with a preserving (or stabilizing) agent to preserve the activity of the test surface. Test surfaces coated with the BRE and the blocking agent may also be coated with the preserving agent. The preserving agent may allow the test surfaces coated with the preserving agent, and the BRE and/or blocking agent, to be stored for an extended period of time before use. Test surfaces coated with the preserving agent, and the BRE and/or blocking agent, may maintain immunological activity for several months compared to if no preserving agent is employed (where immunological activity of a test surface coated with the BRE and/or a blocking agent may continually decline over time).

In some embodiments, the marker being detected, when present in increased or increasing amounts, may indicate a positive/reactive result. In some embodiments, the marker being detected, when absent or present in decreased or decreasing amounts, may indicate a positive/reactive result.

To detect if a marker is present in a sample, a signal from the sample may be compared against the signals of a high standard and a low standard which can be included with the kit or device. A qualitative/visual signal may be generated or visualized of the sample and test standards for making the comparison. The visual indicator may visualize or generate a signal of the sample and standards having a magnitude corresponding to the level of the marker present. The visual indicator may visualize or generate a signal for the first standard consistent with a first level of marker. The visual indicator may visualize a signal for the second standard consistent with a second level of marker.

For example, the visual indicator may visualize for the high standard a signal consistent with a level, such as the minimum level, of the biomarker in a subject with the disease or disorder. The visual indicator may visualize for the low standard a signal consistent with a level, such as the maximum level, of the biomarker in a subject without the disease or disorder. The magnitude of the signal from the biological test sample generated by the visual indicator may be compared against the standards to determine the diagnosis.

Generating the visually detectable signal can be accomplished in several ways. Any visual indicator, including any dye, chromogen, substance, substrate, or solution capable of producing a qualitative indication or visually detectable change may be utilized and included with the kit or device. The generated signal may be visually detectable with or without special equipment. For example, the signal may be a color change, or the generation of a color change along a spectrum, that is visible without special equipment. In some embodiments, it is possible to detect changes in light absorbance visually, with non-specialized light detection equipment, or specialized equipment (e.g., Spectrophotometer). In some embodiments, the signal may be detected by measuring a change in a physical or chemical property of the substrate being tested based on the presence of a label, such as an enzyme label. Types of enzyme-labeled signals known to the art include: light absorbance, light emission, fluorescence, electrochemical signal, pH, etc.

The kits and devices can include instructions for use.

In some embodiments, the kit or device is used to assaying a biological sample, for example a cell sample, such as those discussed above.

Devices that can assist in carrying out the methods disclosed herein are also provided. Included are devices that assist in taking or analyzing biopsies. For example, core needle biopsy instruments, vacuum-assisted biopsy systems, etc.

### V. Methods of Treatment

Any of the disclosed methods of determining whether a subject has a disease or disorder treatable with an active agent that modifies pyruvate metabolism; the TCA cycle; citrate transport or another transporter or enzyme related to formation or cycling of malate, citrate, or acetyl-CoA; glutaminolysis or a transporter or enzyme associated therewith such as glutaminase; or oxidative phosphorylation, selecting the subject for treatment, determining the efficacy of the treatment, and adjusting the treatment dosage and frequency can be coupled with a method of treatment. The methods typically include administering a subject an effective amount of the active agent to prevent, reduce, or treat one or more symptoms of the disease or disorder.

The active agents used in the disclosed methods of treatment are typically those that target a step in energy production concomitant with, or downstream of pyruvate import into the mitochondria by MPC1/2. For example, the agents can target pyruvate metabolism, the tricarboxylic acid cycle, or oxidative phosphorylation, or a related metabolic pathway or cycle including, but not limited to, citrate transport or another transporter or enzyme related to formation or cycling of malate, citrate, or acetyl-CoA; glutaminolysis or a transporter or enzyme associated therewith such as glutaminase.

In preferred embodiments, the modulator is a small molecule, however, the modulator can also be a functional nucleic acid that targets a gene, mRNA, or protein of the metabolic modulators discussed in more detail below. Functional nucleic acids can include, for example, antisense molecules, siRNA, miRNA, piRNA, aptamers, ribozymes, triplex forming molecules, RNAi, external guide sequences, CRISPR/Cas constructs, etc. Any of the active agents, or delivery vehicles thereof, can include a mitochondrial localization signal (MLS) or a protein transduction domain (PTD) to enhance targeting linked, conjugated, or otherwise attached thereto to enhance delivery of the agent into the mitochondrial. PTD and MLS are known in the art, see, for example, U.S. Pat. No. 8,039,587 and WO 2013/103972.

The actual effective amounts of active agent can vary according to factors including the specific modulator administered, the particular composition formulated, the mode of administration, and the age, weight, condition of the subject being treated, as well as the route of administration and the disease or disorder.

In some embodiments, the active agent does not target or otherwise modulate the metabolism of non-target cells or does so at a reduced level compared to target cells. Targets cells are cells exhibiting the metabolism that is the subject of modulation. For example, as discussed in more detail below, in some embodiments, modulators of pyruvate metabolism, the tricarboxylic acid cycle, or oxidative phosphorylation to reverse the Warburg effect in cancer cells and induce cell death. In this case, the target cells are the metabolically dysfunctional cancer cells.

The therapeutic result of the modulator can be compared to a control. Suitable controls are known in the art. A typical control is a comparison of a condition or symptom of a subject prior to and after administration of the active agent. The condition or symptom can be a biochemical, molecular, physiological, or pathological readout. For example, the effect of the composition on a particular symptom, pharmacologic, or physiologic indicator can be compared to an untreated subject, or the condition of the subject prior to treatment. In some embodiments, the symptom, pharmacologic, or physiologic indicator is measured in a subject prior to treatment, and again one or more times after treatment is initiated. In some embodiments, the control is a reference level, or average determined based on measuring the symptom, pharmacologic, or physiologic indicator in one or more subjects that do not have the disease or condition to be treated (e.g., healthy subjects).

A. Treatment Strategies

1. Cancer

The disclosed compositions and methods of treatment thereof are useful in the context of cancer, including tumor therapy. In some embodiments, a subject with cancer is administered an effective amount of an active agent that modifies pyruvate metabolism; the TCA cycle; citrate transport or another transporter or enzyme related to formation or cycling of malate, citrate, or acetyl-CoA; glutaminolysis or a transporter or enzyme associated therewith such as glutaminase; or oxidative phosphorylation to reduce one or more symptoms of the cancer. In some embodiments, the modulator drives an increase in conversion of pyruvate to acetyl-CoA, shifting the metabolism of cancer cells from glycolysis to glucose oxidation and reverses the suppression of mitochondria-dependent apoptosis. In particularly preferred embodiments, the active agent is pyruvate dehydrogenase kinase (PDK) inhibitors such as dichloroacetate, or an analogue or derivative, or conjugate thereof. In some embodiments, the active agent inhibits a downstream step is the TCA cycle or oxidative phosphorylation.

In a mature animal, a balance usually is maintained between cell renewal and cell death in most organs and tissues. The various types of mature cells in the body have a given life span; as these cells die, new cells are generated by the proliferation and differentiation of various types of stem cells. Under normal circumstances, the production of new cells is so regulated that the numbers of any particular type of cell remain constant. Occasionally, though, cells arise that are no longer responsive to normal growth-control mechanisms. These cells give rise to clones of cells that can expand to a considerable size, producing a tumor or neoplasm. A tumor that is not capable of indefinite growth and does not invade the healthy surrounding tissue extensively is benign. A tumor that continues to grow and becomes progressively invasive is malignant. The term cancer refers specifically to a malignant tumor. In addition to uncontrolled growth, malignant tumors exhibit metastasis. In this process, small clusters of cancerous cells dislodge from a tumor, invade the blood or lymphatic vessels, and are carried to other tissues, where they continue to proliferate. In this way a primary tumor at one site can give rise to a secondary tumor at another site.

The compositions and methods described herein are useful for treating subjects having benign or malignant tumors by delaying or inhibiting the growth of a tumor in a subject, reducing the growth or size of the tumor, inhibiting or reducing metastasis of the tumor, and/or inhibiting or reducing symptoms associated with tumor development or growth.

Malignant tumors which may be treated are classified herein according to the embryonic origin of the tissue from which the tumor is derived. Carcinomas are tumors arising from endodermal or ectodermal tissues such as skin or the epithelial lining of internal organs and glands. The disclosed compositions are particularly effective in treating carcinomas. Sarcomas, which arise less frequently, are derived from mesodermal connective tissues such as bone, fat, and cartilage. The leukemias and lymphomas are malignant tumors of hematopoietic ceils of the bone marrow. Leukemias proliferate as single cells, whereas lymphomas tend to grow as tumor masses. Malignant tumors may show up at numerous organs or tissues of the body to establish a cancer.

The types of cancer that can be treated with the provided compositions and methods include, but are not limited to, cancers such as vascular cancer such as multiple myeloma, adenocarcinomas and sarcomas, of bone, bladder, brain, breast, cervical, colo-rectal, esophageal, kidney, liver, lung, nasopharangeal, pancreatic, prostate, skin, stomach, and uterine. In some embodiments, the disclosed compositions are used to treat multiple cancer types concurrently. The compositions can also be used to treat metastases or tumors at multiple locations.

In some embodiments, the cancers are characterized as being triple negative breast cancer, or having one or more KRAS-mutations, EGFR mutations, ALK mutations, RB1 mutations, HIF mutations, KEAP mutations, NRF mutations, or other metabolic-related mutations, or combinations thereof.

2. Inflammatory and Autoimmune Diseases

The disclosed compositions and methods of treatment thereof are also useful in the context of treating inflammatory and autoimmune diseases and disorders. In some embodiments, a subject with an inflammatory, autoimmune, and metabolic disease or disorder is administered an effective amount of an active agent that modifies pyruvate metabolism; the TCA cycle; citrate transport or another transporter or enzyme related to formation or cycling of malate, citrate, or acetyl-CoA; glutaminolysis or a transporter or enzyme associated therewith such as glutaminase; or oxidative phosphorylation to reduce one or more symptoms of the disease or disorder.

Inflammatory immune cells, when activated, exhibit a metabolic profile similar to glycolytic tumor cells. This involves a metabolic shift away from oxidative phosphorylation towards aerobic glycolysis (i.e., the Warburg effect). This switch provides, for example, macrophage with speedy access to ATP and metabolic intermediates for the biosynthesis of immune and inflammatory proteins. A rise in TCA cycle intermediates also occurs, activating HIF-1. Thus, the disclosed methods can be used to modify metabolic pathways in macrophages, dendritic cells, and T cells in subjects suffering from inflammatory, autoimmune, and metabolic diseases. Further, in a glycolytic environment, macrophages polarize to an M2 phenotype which is much more anti-inflammatory and protective of the tumor, this polarization driven primarily by the presence of lactate.

Many of the treatment strategies parallel those utilized in cancer therapy as discussed above. In some embodiments, the modulator drives an increase in conversion of pyruvate to acetyl-CoA, shifting the metabolism of macrophages, dendritic cells, and/or T cells from glycolysis to glucose oxidation and reversing the suppression of mitochondria-dependent apoptosis. In particularly preferred embodiments, the active agent is pyruvate dehydrogenase kinase (PDK) inhibitors such as dichloroacetate, or an analogue or derivative, or conjugate thereof. In some embodiments, the active agent inhibits a downstream step is the TCA cycle or oxidative phosphorylation.

Representative inflammatory or autoimmune diseases and disorders that may be treated include, but are not limited to, rheumatoid arthritis, systemic lupus erythematosus, alopecia areata, anklosing spondylitis, antiphospholipid syndrome, autoimmune Addison's disease, autoimmune hemolytic anemia, autoimmune hepatitis, autoimmune inner ear disease, autoimmune lymphoproliferative syndrome (alps), autoimmune thrombocytopenic purpura (ATP), Behcet's disease, bullous pemphigoid, cardiomyopathy, celiac sprue-dermatitis, chronic fatigue syndrome immune deficiency, syndrome (CFIDS), chronic inflammatory demyelinating polyneuropathy, cicatricial pemphigoid, cold agglutinin disease, Crest syndrome, Crohn's disease, Dego's disease, dermatomyositis, dermatomyositis—juvenile, discoid lupus, essential mixed cryoglobulinemia, fibromyalgia-fibromyositis, grave's disease, guillain-barre, hashimoto's thyroiditis, idiopathic pulmonary fibrosis, idiopathic thrombocytopenia purpura (ITP), Iga nephropathy, insulin dependent diabetes (Type I), juvenile arthritis, Meniere's disease, mixed connective tissue disease, multiple sclerosis, myasthenia gravis, pemphigus vulgaris, pernicious anemia, polyarteritis nodosa, polychondritis, polyglancular syndromes, polymyalgia rheumatica, polymyositis and dermatomyositis, primary agammaglobulinemia, primary biliary cirrhosis, psoriasis, Raynaud's phenomenon, Reiter's syndrome, rheumatic fever, sarcoidosis, scleroderma, Sjogren's syndrome, stiff-man syndrome, Takayasu arteritis, temporal arteritis/giant cell arteritis, ulcerative colitis, uveitis, vasculitis, vitiligo, and Wegener's granulomatosis.

3. Modulating the Warburg Effect

The disclosed compositions and methods of treatment thereof are useful for modulating the Warburg effect. For example, in some embodiments, an effective amount of an active agent that modifies pyruvate metabolism, the TCA cycle, or oxidative phosphorylation is administered to a subject to reverse the Warburg effect. Such compositions and methods can be effective, for example, when the subject has a disease or disorder characterized by cells exhibiting Warburg metabolism, such as cancer cells or immune cells. The active agent can be, for example, one that shifts the metabolism of the cells from glycolysis to glucose oxidation, reverses the suppression of mitochondria-dependent apoptosis, increases the oxidation of pyruvate, reduces the conversion of pyruvate to lactate, or a combination thereof.

In other embodiments, an effective amount of an active agent that modifies pyruvate metabolism; the TCA cycle; citrate transport or another transporter or enzyme related to formation or cycling of malate, citrate, or acetyl-CoA; glutaminolysis or a transporter or enzyme associated therewith such as glutaminase; or oxidative phosphorylation is administered to a subject to reverse the Inverse Warburg effect. As introduced above, the Inverse Warburg effect occurs when metabolic reprogramming leads to the up-regulation of oxidative phosphorylation (OXPHOS) in mitochondria of certain cells. The Inverse Warburg effect has been characterized as a compensatory increase in OXPHOS designed to maintain adequate energy production, and has been identified a hallmark of neurodegenerative disease progression and a complication of diabetes (Demetrius, et al., Biogerontology, 13(6):583-94 (2012), Demetrius, et al., Front Physiol, 5: 522 (2015), Craft, et al., Arch Neurol. 69(1):29-38 (2012)).

For example an exemplary method can include increasing PDK in cells of a subject by increasing or stabilizing HIF1α (e.g., by administering the subject an iron chelator) which in turn will drive a more glycolytic phenotype and preserve cells such as neurons or cardiac cells.

Exemplary neurodegenerative diseases include, but are not limited to, Huntington's Disease (HD), Amyotrophic Lateral Sclerosis (ALS), Parkinson's Disease (PD) and PD-related disorders, Alzheimer's Disease (AD) and other dementias, Prion Diseases such as Creutzfeldt-Jakob Disease, Corticobasal Degeneration, Frontotemporal Dementia, HIV-Related Cognitive Impairment, Mild Cognitive Impairment, Motor Neuron Diseases (MND), Spinocerebellar Ataxia (SCA), Spinal Muscular Atrophy (SMA), Friedreich's Ataxia, Lewy Body Disease, Alpers' Disease, Batten Disease, Cerebro-Oculo-Facio-Skeletal Syndrome, Corticobasal Degeneration, Gerstmann-Straussler-Scheinker Disease, Kuru, Leigh's Disease, Monomelic Amyotrophy, Multiple System Atrophy, Multiple System Atrophy With Orthostatic Hypotension (Shy-Drager Syndrome), Multiple Sclerosis (MS), Neurodegeneration with Brain Iron Accumulation, Opsoclonus Myoclonus, Posterior Cortical Atrophy, Primary Progressive Aphasia, Progressive Supranuclear Palsy, Vascular Dementia, Progressive Multifocal Leukoencephalopathy, Dementia with Lewy Bodies, Lacunar syndromes, Hydrocephalus, Wernicke-Korsakoffs syndrome, post-encephalitic dementia, cancer and chemotherapy-associated cognitive impairment and dementia, and depression-induced dementia and pseudodementia.

Other diseases and disorders that may be characterized by cell exhibiting the Inverse Warburg effect include, but are not limited to, neurological disorders, seizure disorders, cardiovascular disease, ischemia, and endometriosis. For example, active agents can, for example, down regulate the activity of the PDC, decrease the oxidation of pyruvate in mitochondria, increase the conversion of pyruvate to lactate in the cytosol, reduce apoptosis, or a combination thereof. Thus, such treatment strategies can be used to divert cellular metabolism from aerobic glycolysis, increase cellular longevity, or a combination thereof.

Thus in some embodiments, a subject with a condition or disorder selected from neurodegenerative disease or disorder, diabetes, a neurological disorder, seizure disorder, cardiovascular disease, ischemia, and endometriosis is administered an effective amount of an active agent that modifies pyruvate metabolism, the TCA cycle, or oxidative phosphorylation to reduce one or more symptoms of the condition or disorder.

B. Exemplary Active Agents

As introduced above, the preferred active agents typically act on a metabolic target concomitant with or downstream of pyruvate import into the mitochondria. The active agents typically increase or decrease pyruvate metabolism, the TCA cycle, or oxidative phosphorylation in cells in a subject in need thereof.

1. Pyruvate Dehydrogenase Kinase

The agent can be one that directly or indirectly activates or inhibits pyruvate dehydrogenase (PDH), or activates or inhibits pyruvate dehydrogenase kinase (PDK). PDH is the first component enzyme of pyruvate dehydrogenase complex. As discussed above, the pyruvate dehydrogenase complex converts cytosolic pyruvate to mitochondrial acetyl-CoA, the substrate for the Krebs' cycle. Pyruvate dehydrogenase kinase (PDK) is a mitochondrial enzyme that is activated in a variety of cancers and results in the selective inhibition of PDH.

Inhibition of PDK with either small interfering RNAs or the orphan drug dichloroacetate (DCA) shifts the metabolism of cancer cells from glycolysis to glucose oxidation and reverses the suppression of mitochondria-dependent apoptosis. In addition, the inhibition of PDK increases the production of diffusible Krebs' cycle intermediates and mitochondria-derived reactive oxygen species, activating p53 or inhibiting pro-proliferative and pro-angiogenic transcription factors like nuclear factor of activated T cells and hypoxia-inducible factor 1α. DCA is a small 150 Da molecule that can penetrate cell membranes and most tissues, including the brain. DCA activates PDH by inhibiting PDK at a concentration of 10-250 μM, in a dose-dependent fashion (Stacpoole, 1989). There are four PDK isoforms that are expressed in most tissues with the most sensitive to DCA being PDK2.

In particularly preferred embodiments, the active agent is DCA or a derivative, analogue, or conjugate thereof. A recent study identified a mitochondria-targeted DCA analogue with a much improved cellular and mitochondrial uptake (Pathak R K et al., ACS Chem. Biol., 9 (5) 1178-1187 (2014)). The compound uses a lipophilic triphenylphosphonium (TPP) cation moiety for the targeted delivery and accumulation into the mitochondrial matrix. The study showed that the compound efficiently reduced glycolytic functions, reduced basal cellular respiration, suppressed the calculated ATP synthesis, and attenuated the spare respiratory capacity in prostate cancer cells (Pathak R K et al., ACS Chem. Biol., 9 (5) 1178-1187 (2014)). In particularly preferred embodiments, the active agent is a mitochondria-targeted DCA analogue.

Additional examples of metabolic inhibitors such as DCA or analogue or derivative thereof and a mitochondria targeting moiety such as triphenylphosphonium cation are disclosed in WO/2015/002996, which is specifically incorporated by reference in its entirety.

Oxythiamine is a thiamine antagonist and inhibits transketolase and pyruvate dehydrogenase, which require thiamine pyrophosphate (TPP) as a cofactor for their enzyme activity. Early studies suggest that oxythiamine is phosphorylated to yield diphosphate ester which then acts as a strong competitive inhibitor (K_{I}=0.07 μM) against the normal cofactor TPP (Kₘ=0.11 μM) when highly purified pyruvate dehydrogenase was used (Strumilo S A et al., Biomed Biochim Acta 43:159-163 (1984)).

2. Tricarboxylic Acid (TCA) Cycle In some embodiments, the active agent targets the tricarboxylic acid (TCA) cycle. Therefore, in some embodiments, the active agent is one that directly or indirectly inhibits, activates, or enhances the TCA cycle, or a component thereof. Examples of suitable targets include, but are not limited to, succinate dehydrogenase, isocitrate dehydrogenase, aconitase etc.

Arsenic trioxide (ATO), a mitochondrial toxicant, is currently used in the treatment of acute promyelocytic leukemia (APL). ATO has several mechanisms by which APL is targeted. Mutations of isocitrate dehydrogenase (IDH), another component of the TCA cycle, are frequently found in several types of cancer such as glioma and acute myeloid leukemia. Inhibitors (e.g. AGI-5198) of IDH mutants by Rohle et al. have been developed and demonstrated anti-cancer activities (Rohle D et al., Science. 340:626-630 (2013)).

3. Electron Transport Chain

In some embodiments, the active agent is one that directly or indirectly inhibits, activates, or enhances the electron transport chain, or a component thereof. Suitable modulators include classic Complex I-IV inhibitors and ATP synthase inhibitors. Exemplary inhibitors of the ETC are amytal, rotenone, antimycin A, CO, sodium azide, and cyanides. In a particular example, the modulator is metformin. Metformin is a clinically approved drug by the FDA to treat type II diabetes, targets the mitochondrial complex I and thereby reducing ATP synthesis.

4. Citrate Transport

Fatty acid synthesis, which occurs in the cytosol requires acetyl-CoA. Typically, in normal cells, intra-mitochondrial acetyl-CoA first reacts with oxaloacetate to form citrate in the TCA cycle, catalyzed by citrate synthase. Citrate then passes into the cytosol through the citrate transporter, where it is cleaved by citrate lyase to regenerate acetyl-CoA. If pyruvate transport into the mitochondria is low or absent, for example because MPC expression is reduced or absent, it converts itself to malate which is exchanged at the citrate transporter for citrate and thus can continue through to ox/phos while also acetylating in the cytosol. Thus, in some embodiments, a subject is selected for treatment, and optionally treated, with an active agent that reduces or inhibits citrate transport or another transporter or enzyme related to formation or cycling of malate, citrate, or acetyl-CoA, particularly if MPC expression is reduced or absent (e.g., negative/low/mutant (loss of function)).

In some embodiments, the target is a citrate transporter such as citrate transport protein (CTP) (also referred to as tricarboxylate transport protein and SLC25A1). The Examples below show that a citrate transporter inhibitor is particularly deadly to cells with reduced MPC expression. An exemplary citrate transporter inhibitor is Mitochondrial Citrate Transport Protein (CTP) Inhibitor (also referred to as CAS 412940-35-3, and 4-chloro-3-{[(3-nitrophenyl)amino]sulfonyl}benzoic acid). See, e.g., EDM Millipore catalogue number 475877, and Aluvila, S., et al. Mol. Pharmacol. 77, 26 (2010); and Sun, et al., Mol Cell Pharmacol, 2(3):101-110 (2010), which describes other citrate transporter inhibitors, all of which are specifically incorporated by reference herein in their entireties.

Other particularly preferred targets include, but are not limited to ATP citrate lyase and acyl-CoA synthase. Exemplary active agents include, but are not limited to, ATP Citrate Lyase Inhibitors such as BMS 303141, MEDICA 16, and SB 204990, and acyl-CoA synthase inhibitors such as enoximone and triacsins.

5. Glutaminolysis

Glutaminolysis takes place in all proliferating cells, including lymphocytes, thymocytes, colonocytes, adipocytes and especially in tumor cells. In tumor cells the TCA cycle can be truncated and phosphate dependent glutaminase and NAD(P)-dependent malate decarboxylase can be overexpressed, which in combination can lead to an alternative form of energy production through the degradation of the amino acid glutamine to glutamate, aspartate, pyruvate CO2, lactate and citrate. Thus, in addition to glycolysis, glutaminolysis can server as a second form of energy production in cancer cells. High extracellular glutamine concentrations can stimulate tumor growth and are important for cell transformation, while a reduction of glutamine correlates with phenotypical and functional differentiation of the cells.

When MPC expression is reduced or absent, cells rely more on glutaminolysis. Thus, inhibitors of glutaminolysis or a transporter or enzyme associated therewith such as glutaminase will work best.

Thus, in some embodiments, a subject is selected for treatment, and optionally treated, with an active agent that reduces or inhibits glutaminolysis or a transporter or enzyme associated therewith such as glutaminase, particularly if MPC expression is reduced or absent (e.g., negative/low/mutant (loss of function)). The Examples below show that glutaminase inhibition is particularly deadly to cells with reduced MPC expression.

Alternatively, if MPC is not significantly reduced (e.g., positive/high/mutant (gain of function)) AND HIF1 or HIF2 levels are high cancer cells may also rely much more so on glutaminolysis and thus an inhibitor of this pathway would be much more effective than say if the HIF1 or HIF2 levels were low OR if MPC was negative (where the cell has already evolved an alternative pathway). Hypoxia-inducible factor 1 (HIF1) or Hypoxia-inducible factor 2 (HIF2) essentially block acetyl coA production in MPC(+) cell lines leaving the cell to rely more on glutaminolysis. Thus, in some embodiments, a subject is selected for treatment, and optionally treated, with an active agent that reduces or inhibits glutaminolysis or a transporter or enzyme associated therewith such as glutaminase, if MPC expression is not significantly reduced (e.g., positive/high/mutant (gain of function)) AND HIF1 or HIF2 levels are not substantially reduces, near wildtype (or control), or higher than wildtype (or control). The Examples below show that glutaminase inhibition is particularly deadly to MPC-positive, HIF1 “high” expressing cells, relative to HIF1 “low” expressing cells.

Preferred targets include, for example, glutaminase, glutamate dehydrogenase, and isocitrate dehydrogenase (IDH) 1 and 2.

Exemplary active agents include, but are not limited to, inhibitors such as BPTES, CB-839, 968, EGCG, AG-120, AG-221.

6. Other Exemplary Active Agents Other active agents include, for example, UK5099 as an MPC blocker which would drive more of a glycolytic phenotype. HIF-1α activators or iron chelators as HIF1α activators/stabilizers can be used to increase PDK. HIF-1α activators are known in the art and include, but are not limited to, natural product-derived small molecules such as those described in Nagle, et al., Curr Pharm Des. 2006; 12(21): 2673-2688, which is specifically incorporated by reference herein in its entirety. Another example of a HIF-1α activator is deferoxamine (Guo, et al., Exp Neural., 280:13-23 (2016)).

In some embodiments, the active agent is one that increases ATP.

C. Formulations

The formulations and pharmaceutical compositions containing an effective amount of the disclosed composition in a pharmaceutical carrier appropriate for administration to an individual in need thereof to treat one or more symptoms of a disease or disorder are also provided. The formulations can be administered parenterally (e.g., by intramuscular, intraperitoneal, intravenous (IV) or subcutaneous injection or infusion). It may also be possible to administer topically (e.g., to a mucosal surface such as the mouth, lungs, intranasal, intravaginally, etc.). The compositions can be administered locally or systemically.

Drugs can be formulated for immediate release, extended release, or modified release. A delayed release dosage form is one that releases a drug (or drugs) at a time other than promptly after administration. An extended release dosage form is one that allows at least a twofold reduction in dosing frequency as compared to that drug presented as a conventional dosage form (e.g. as a solution or prompt drug-releasing, conventional solid dosage form). A modified release dosage form is one for which the drug release characteristics of time course and/or location are chosen to accomplish therapeutic or convenience objectives not offered by conventional dosage forms such as solutions, ointments, or promptly dissolving dosage forms. Delayed release and extended release dosage forms and their combinations are types of modified release dosage forms.

Formulations are prepared using a pharmaceutically acceptable “carrier” composed of materials that are considered safe and effective and may be administered to an individual without causing undesirable biological side effects or unwanted interactions. The “carrier” is all components present in the pharmaceutical formulation other than the active ingredient or ingredients.

In some embodiments, the active agent is incorporated into or encapsulated by a nanoparticle, microparticle, micelle, synthetic lipoprotein particle, or carbon nanotube. For example, the compositions can be incorporated into a vehicle such as polymeric microparticles which provide controlled release of the active agent. In some embodiments, release of the drug(s) is controlled by diffusion of the active agent out of the microparticles and/or degradation of the polymeric particles by hydrolysis and/or enzymatic degradation. Suitable polymers include ethylcellulose and other natural or synthetic cellulose derivatives. Polymers which are slowly soluble and form a gel in an aqueous environment, such as hydroxypropyl methylcellulose or polyethylene oxide may also be suitable as materials for drug containing microparticles. Other polymers include, but are not limited to, polyanhydrides, poly (ester anhydrides), polyhydroxy acids, such as polylactide (PLA), polyglycolide (PGA), poly(lactide-co-glycolide) (PLGA), poly-3-hydroxybutrate (PHB) and copolymers thereof, poly-4-hydroxybutyrate (P4HB) and copolymers thereof, polycaprolactone and copolymers thereof, and combinations thereof.

The active agent can be incorporated into or prepared from materials which are insoluble in aqueous solution or slowly soluble in aqueous solution, but are capable of degrading within the GI tract by means including enzymatic degradation, surfactant action of bile acids, and/or mechanical erosion. As used herein, the term “slowly soluble in water” refers to materials that are not dissolved in water within a period of 30 minutes. Preferred examples include fats, fatty substances, waxes, waxlike substances and mixtures thereof. Suitable fats and fatty substances include fatty alcohols (such as lauryl, myristyl stearyl, cetyl or cetostearyl alcohol), fatty acids and derivatives, including, but not limited to, fatty acid esters, fatty acid glycerides (mono-, di- and tri-glycerides), and hydrogenated fats. Specific examples include, but are not limited to hydrogenated vegetable oil, hydrogenated cottonseed oil, hydrogenated castor oil, hydrogenated oils available under the trade name Sterotex®, stearic acid, cocoa butter, and stearyl alcohol. Suitable waxes and wax-like materials include natural or synthetic waxes, hydrocarbons, and normal waxes.

Specific examples of waxes include beeswax, glycowax, castor wax, carnauba wax, paraffins and candelilla wax. As used herein, a wax-like material is defined as any material which is normally solid at room temperature and has a melting point of from about 30 to 300° C.

Parenteral Formulations

The composition can be formulated for parenteral delivery, such as injection or infusion, in the form of a solution or suspension, or a powder. The formulation can be administered via any route, such as, the blood stream or directly to the organ or tissue to be treated. The particles may be provided in a lyophilized or dried form in a unit dosage form, for suspension at the time of injection. These may be provided in a kit with an appropriate amount of diluent such as sterile water or buffered solution.

Parenteral formulations can be prepared as aqueous compositions using techniques known in the art. Typically, such compositions can be prepared as injectable formulations, for example, solutions or suspensions; solid forms suitable for using to prepare solutions or suspensions upon the addition of a reconstitution medium prior to injection; emulsions, such as water-in-oil (w/o) emulsions, oil-in-water (o/w) emulsions, and microemulsions thereof, liposomes, or emulsomes.

The carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof. The proper fluidity can be maintained, for example, by the use of a coating, such as lecithin, by the maintenance of the required particle size in the case of dispersion and/or by the use of surfactants. In many cases, it will be preferable to include isotonic agents, for example, sugars or sodium chloride.

Solutions and dispersions of the compounds or nanoparticles can be prepared in water or another solvent or dispersing medium suitably mixed with one or more pharmaceutically acceptable excipients including, but not limited to, surfactants, dispersants, emulsifiers, pH modifying agents, and combination thereof.

Suitable surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-β-alanine, sodium N-lauryl-β-iminodipropionate, myristoamphoacetate, lauryl betaine and lauryl sulfobetaine.

The formulation can contain a preservative to prevent the growth of microorganisms. Suitable preservatives include, but are not limited to, parabens, chlorobutanol, phenol, sorbic acid, and thimerosal. The formulation may also contain an antioxidant to prevent degradation of the active agent(s) or nanoparticles.

The formulation is typically buffered to a pH of 3-8 for parenteral administration upon reconstitution. Suitable buffers include, but are not limited to, phosphate buffers, acetate buffers, and citrate buffers.

Water soluble polymers are often used in formulations for parenteral administration. Suitable water-soluble polymers include, but are not limited to, polyvinylpyrrolidone, dextran, carboxymethylcellulose, and polyethylene glycol.

Sterile injectable solutions can be prepared by incorporating the compound or nanoparticles in the required amount in the appropriate solvent or dispersion medium with one or more of the excipients listed above, as required, followed by filtered sterilization. Generally, dispersions are prepared by incorporating the various sterilized compositions into a sterile vehicle which contains the basic dispersion medium and the required other ingredients from those listed above. In the case of sterile powders for the preparation of sterile injectable solutions, the preferred methods of preparation are vacuum-drying and freeze-drying techniques which yield a powder of the compound or nanoparticle plus any additional desired ingredient from a previously sterile-filtered solution thereof. The powders can be prepared in such a manner that the particles are porous in nature, which can increase dissolution of the particles. Methods for making porous particles are well known in the art.

Pharmaceutical formulations for parenteral administration are preferably in the form of a sterile aqueous solution or suspension of particles formed from one or more polymer-drug conjugates. Acceptable solvents include, for example, water, Ringer's solution, phosphate buffered saline (PBS), and isotonic sodium chloride solution. The formulation may also be a sterile solution, suspension, or emulsion in a nontoxic, parenterally acceptable diluent or solvent such as 1,3-butanediol.

In some instances, the formulation is distributed or packaged in a liquid form. Alternatively, formulations for parenteral administration can be packed as a solid, obtained, for example by lyophilization of a suitable liquid formulation. The solid can be reconstituted with an appropriate carrier or diluent prior to administration.

Solutions, suspensions, or emulsions for parenteral administration may be buffered with an effective amount of buffer necessary to maintain a pH suitable for ocular administration. Suitable buffers are well known by those skilled in the art and some examples of useful buffers are acetate, borate, carbonate, citrate, and phosphate buffers.

Solutions, suspensions, or emulsions for parenteral administration may also contain one or more tonicity agents to adjust the isotonic range of the formulation. Suitable tonicity agents are well known in the art. Examples include glycerin, mannitol, sorbitol, sodium chloride, and other electrolytes.

Solutions, suspensions, or emulsions for parenteral administration may also contain one or more preservatives to prevent bacterial contamination of the ophthalmic preparations. Suitable preservatives are known in the art, and include polyhexamethylenebiguanidine (PHMB), benzalkonium chloride (BAK), stabilized oxychloro complexes (otherwise known as Purite®), phenylmercuric acetate, chlorobutanol, sorbic acid, chlorhexidine, benzyl alcohol, parabens, thimerosal, and mixtures thereof.

Solutions, suspensions, or emulsions for parenteral administration may also contain one or more excipients known art, such as dispersing agents, wetting agents, and suspending agents.

D. Treatment Regimen

As discussed herein, the disclosed methods can be used to adjust dosage and frequency of administration. In some embodiments, dosages are administered once, twice, or three times daily, or every other day, two days, three days, four days, five days, or six days to a human. In some embodiments, dosages are administered about once or twice every week, every two weeks, every three weeks, or every four weeks. In some embodiments, dosages are administered about once or twice every month, every two months, every three months, every four months, every five months, or every six months.

In some embodiments, the regimen includes one or more cycles of a round of therapy followed by a drug holiday (e.g., no drug). The round of the therapy can be, for example, and of the administrations discussed above. Likewise, the drug holiday can be 1, 2, 3, 4, 5, 6, or 7 days; or 1, 2, 3, 4 weeks, or 1, 2, 3, 4, 5, or 6 months.

Particular dosage regimens include, for example, one or more cycles in which the subject is administered the drug each of five days in a row, followed by a two-day drug holiday.

E. Combination Therapies

The disclosed compositions can be administered alone or in combination with one or more conventional therapies, for example, a conventional therapy for the disease or disorder being treated. In some embodiments, the conventional therapy includes administration of one or more of the disclosed compositions in combination with one or more additional active agents. The combination therapies can include administration of the active agents together in the same admixture, or in separate admixtures. Therefore, in some embodiments, the pharmaceutical composition includes two, three, or more active agents. Such formulations typically include an effective amount of a modulator of cancer cell metabolism. The additional active agent(s) can have the same, or different mechanisms of action. In some embodiments, the combination results in an additive effect on the treatment of the cancer. In some embodiments, the combinations result in a more than additive effect on the treatment of the disease or disorder.

For example, additional therapeutic agents include conventional cancer therapeutics such as chemotherapeutic agents, cytokines, chemokines, and radiation therapy. The majority of chemotherapeutic drugs can be divided into: alkylating agents, antimetabolites, anthracyclines, plant alkaloids, topoisomerase inhibitors, and other antitumour agents. All of these drugs affect cell division or DNA synthesis and function in some way. Additional therapeutics include monoclonal antibodies and the tyrosine kinase inhibitors e.g., imatinib mesylate (GLEEVEC® or GLIVEC®), which directly targets a molecular abnormality in certain types of cancer (chronic myelogenous leukemia, gastrointestinal stromal tumors).

Representative chemotherapeutic agents include, but are not limited to, amsacrine, bleomycin, busulfan, capecitabine, carboplatin, carmustine, chlorambucil, cisplatin, cladribine, clofarabine, crisantaspase, cyclophosphamide, cytarabine, dacarbazine, dactinomycin, daunorubicin, docetaxel, doxorubicin, epipodophyllotoxins, epirubicin, etoposide, etoposide phosphate, fludarabine, fluorouracil, gemcitabine, hydroxycarb amide, idarubicin, ifosfamide, innotecan, leucovorin, liposomal doxorubicin, liposomal daunorubici, lomustine, mechlorethamine, melphalan, mercaptopurine, mesna, methotrexate, mitomycin, mitoxantrone, oxaliplatin, paclitaxel, pemetrexed, pentostatin, procarbazine, raltitrexed, satraplatin, streptozocin, teniposide, tegafur-uracil, temozolomide, teniposide, thiotepa, tioguanine, topotecan, treosulfan, vinblastine, vincristine, vindesine, vinorelbine, taxol and derivatives thereof, trastuzumab (HERCEPTIN®), cetuximab, and rituximab (RITUXAN® or MABTHERA®), bevacizumab (AVASTIN®), and combinations thereof. Representative pro-apoptotic agents include, but are not limited to, fludarabinetaurosporine, cycloheximide, actinomycin D, lactosylceramide, 15d-PGJ(2)5 and combinations thereof.

### EXAMPLES

### Example 1

### Efficacy of PDK Inhibitor Positively Correlates with MPC Expression Levels

Materials and Methods

KULA2 refers to a dichloroacetate (DCA) analogue targeted to the mitochondria with a lipophilic triphenylphosphonium (TPP) cation moiety ((Pathak R K et al., ACS Chem. Biol., 9 (5) 1178-1187 (2014), (Pathak R K et al., ACS Chem. Biol., 9 (5) 1178-1187 (2014), and WO/2015/002996).

HCT116 represents the MPC null group, having minimal to no expression of MPC1 or MPC2. CT26 is the MPC+ group. MPC levels were verified via western blotting or otherwise known (see, e.g., Schell, et al., Molecular Cell, 56:400-413 (2014)).

In both models HCT116 and CT26 the mice (between 8-10 per arm) were dosed with KULA2 via i.p. injection and vehicle, HCT116 tumors at a dose of 20 mg/kg, and CT26 tumors at a dose of 18 mg/kg. Dosing schedule in both cases was 5 days on, 2 days off for the duration of the study.

Results

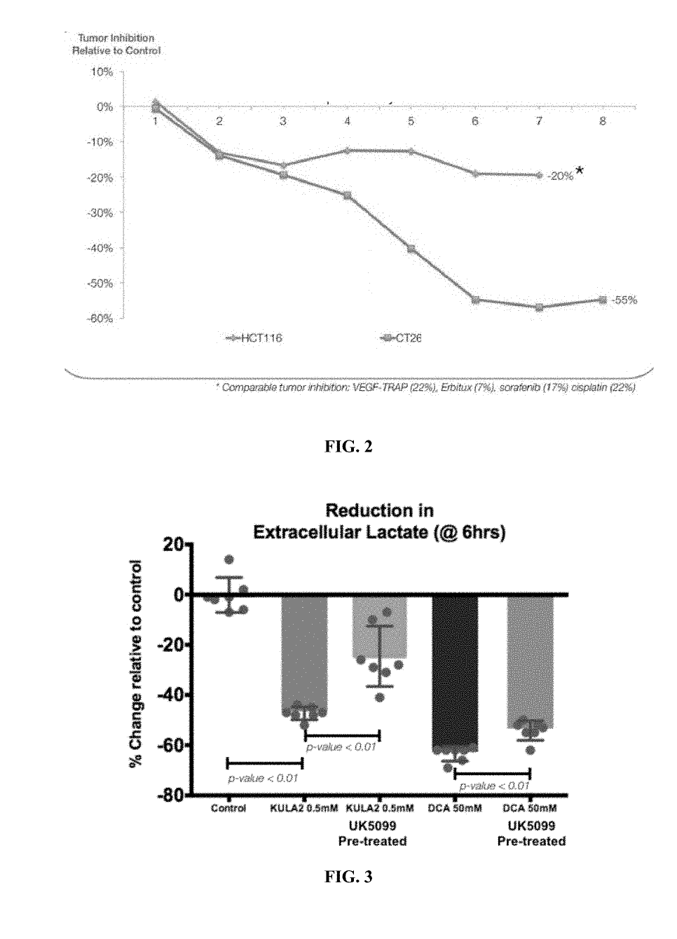

Experiments were designed to determine if MPC expression correlates with the efficacy of PDK inhibitor. A PDK inhibitor, KULA2, was administered to mice harboring HCT116 tumors (at a dose of 20 mg/kg) or CT26 tumors (at a dose of 18 mg/kg). MPC1 and MPC2 expression is substantially reduced in HCT116 cells, but not CT26 cells. The results, presented in FIG. 2, show that KULA2 inhibits tumors by about 20%, a result comparable to VEGF-TRAP (22%), Erbitux (7%), sorafenib (17%), and cisplatin (22%). However in the CT26 MPC positive model, KULA2 inhibits tumors by about 55%. The results indicate that PDK inhibitor efficacy can positively correlate with MPC expression in tumor cells. The results also indicate that MPC expression can used as a biomarker to select subjects for treatment with KULA2, other PDK inhibitors, and other modulators to the TCA and oxidative phosphorylation downstream of pyruvate import into the mitochondria.

### Example 2

### Inhibition of MPC1 and MPC2 with a Known MPC Blocker Lowers the Lactate Reduction Normally Seen with PDK Inhibitors

Materials and Methods

A549 cells were pre-treated for 16 hrs with the MPC inhibitor UK5099 and then subsequently treated with the test articles for 6 hrs; extracellular lactate was then measured. Dichloroacetate (DCA) and KULA2, a dichloroacetate (DCA) analogue targeted to the mitochondria with a lipophilic triphenylphosphonium (TPP) cation moiety, were used ((Pathak R K et al., ACS Chem. Biol., 9 (5) 1178-1187 (2014), (Pathak R K et al., ACS Chem. Biol., 9 (5) 1178-1187 (2014), and WO/2015/002996). DCA is dosed at 50 mM and KULA2 is dosed at 500 μM.

Results

Experiments were designed to determine if the presence or absence (inhibition) of MPC affects the level of extracellular lactate following treatment with a PDK inhibitor. The results in FIG. 3 show that inhibition of MPC1 and MPC2 with UK5099, a known MPC blocker, lowers the lactate reduction normally seen with PDK inhibitors; more so for more targeted inhibitors that rely on being downstream of MPC1 and MPC2. In other words, PDK inhibitors are more effective in the presence of functional MPC, as evident by a greater reduction in cellular lactate. These results also illustrate that extracellular lactate can serve as a biomarker for measuring PDK inhibitor efficacy.

### Example 3

### Inhibition of Citrate Transporter Reduces Cell Viability in Cells with Reduced Expression of MPC

Materials and Methods

MPC-positive A549 cells (*) and MPC-negative HCT116 cells (#) were treated with various doses (μM) of a citrate transporter inhibitor.

Results

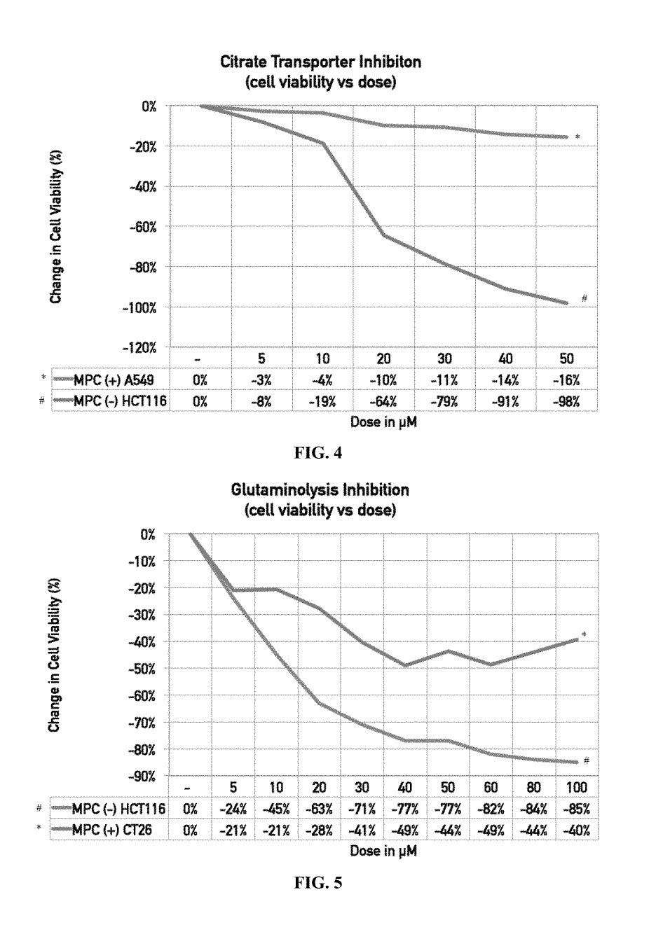

The results are illustrated in FIG. 4, a line graph showing the change in cell viability (%) of MPC-positive A549 cells (*) and MPC-negative HCT116 cells (#) following treatment with increasing doses (μM) of the citrate transporter inhibitor. The results indicate that MPC status can be used to predict efficacy (responders) of treatment with citrate transport inhibitors (or anything in that path including citrate lysate and acyl-coa synthase). More particularly, negative/low/mutant (loss of function) MPC means that citrate pathway inhibition will be successful. If MPC is positive/high/mutant (gain of function) there might be much less of a therapeutic effect.

### Example 4

### Inhibition of Glutaminolysis Reduces Cell Viability in Cells with Reduced Expression of MPC and/or High HIF1 Level

Materials and Methods

MPC-positive A549 cells and MPC-negative HCT116 cells were treated with various doses (μM) of a glutaminolysis inhibitor.

HIF1-High, MPC-positive A549 cells and HIF1-Low, MPC-positive CT26 cells were treated with various doses (μM) of a glutaminolysis inhibitor.

Results

FIG. 5 in a line graph showing the change in cell viability (%) of MPC-positive A549 cells (*) and MPC-negative HCT116 cells (#) following treatment with increasing doses (μM) of the glutaminolysis inhibitor. The results in FIG. 5 indicate that MPC status can be used for predicting efficacy (responders) of treatment with inhibitors of glutaminolysis including inhibitors of glutaminase (GLS). More particularly, when MPC is negative cells rely more on glutaminolysis and so these inhibitors will work best.

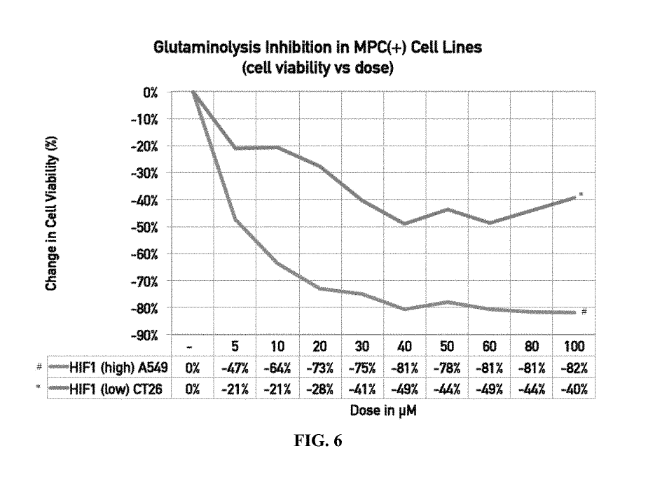

FIG. 6 is a line graph showing the change in cell viability (%) of HIF1-low, MPC-positive CT26 cells (*) and HIF1-high, MPC-positive A549 cells (#) following treatment with increasing doses (μM) of a glutaminolysis inhibitor. The results in FIG. 6 show that when MPC is positive/high/mutant (gain of function) AND HIF1 or HIF2 levels are high then cell, including cancer cells, are more likely to rely much more so on glutaminolysis. Thus an inhibitor of this pathway would be much more effective than if the HIF1 or HIF2 levels were low OR if MPC was negative (where the cell has already evolved an alternative pathway). HIF1 or HIF2 essentially block acetyl coA production in MPC(+) cell lines leaving the cell to rely more on glutaminolysis.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

I claim:

1. A method of selecting and treating a subject for a disease or disorder comprising:
(a) detecting the level of one or more biomarkers selected from the group consisting of one or more Mitochondrial Pyruvate Carriers (MPC), one or more components of the Pyruvate Dehydrogenase Complex (PDC), and one or more glutamine transporters in diseased or disordered cells obtained from the subject;
(b) selecting the subject for treatment if the subject meets criteria comprising: the level of the biomarker in the disease or disordered cells is at least 20%, 25%, 30%, 35%, 40%, 45%, 50%, 55%, 60%, 65%, 70%, 75%, 80%, 85%, 90%, 95%, 100%, or more than 100% relative to a control; and
(c) administering the selected subjects an effective amount of an active agent that modifies pyruvate metabolism; the TCA cycle; citrate transport or another transporter or enzyme related to formation or cycling of malate, citrate, or acetyl-CoA; glutaminolysis or a transporter or enzyme associated therewith such as glutaminase; or oxidative phosphorylation to treat the disease or disorder.

2. The method of claim 1, wherein the MPC is selected from Mitochondrial Pyruvate Carrier 1, Mitochondrial Pyruvate Carrier 2, and the combination thereof.

3. The method of claim 1 wherein the component of the PDC is pyruvate dehydrogenase subunit α, pyruvate dehydrogenase subunit β, dihydrolipoyl transacetylase, dihydrolipoyl dehydrogenase, and combinations thereof.

4. The method of claim 1, wherein the biomarker is mitochondrial glutamine transporter, and the criteria under step (b) further comprises low, substantially no, or no expression of MPC1, MPC2, or a combination thereof.

5. The method of claim 1, wherein the disease or disorder is cancer or inflammatory or autoimmune disease or disorder.

6. The method of claim 1, wherein the active agent is selected from the group consisting of a pyruvate dehydrogenase kinase inhibitor, an inhibitor of the tricarboxylic acid (TCA) cycle, or an inhibitor of the electron transport chain; an of inhibitor citrate transport or another transporter or enzyme related to formation or cycling of malate, citrate, or acetyl-CoA; or an inhibitors of glutaminolysis or a transporter or enzyme associated therewith such as glutaminase

7. The method claim 1 wherein the level of pyruvate dehydrogenase kinase is increased in the diseased or disordered cells or hypoxia-inducible factor-1α (HIF-1α) is increased in a biological sample relative to the control and is positively correlated with the level of pyruvate dehydrogenase kinase in the diseased or disordered cells or hypoxia-inducible factor-1α (HIF-1α) is increased in a biological sample.

8. The method of claim 7, wherein the pyruvate dehydrogenase kinase inhibitor is dichloroacetate, or an analogue, derivative, or conjugate thereof, optionally targeted to the mitochondria.

9. The method of claim 7, wherein the subject is administered a less than standard dosing regimen of DCA or analogue, derivative, or conjugate thereof if the subject has at least one KGM allele, has at least one EGM allele, does not have at least one EGT allele, or a combination thereof at amino acid positions 32, 42, and 82 of the GSTz1/MAAI protein.

10. The method of claim 1, wherein the level of pyruvate dehydrogenase kinase is not substantially increased, is equal to, or is lower in the diseased or disordered cells or hypoxia-inducible factor-1α (HIF-1α) is not substantially increased, is equal to, or is lower in a biological sample relative to the control, and optionally the active agent is one that acts downstream of PDK such as an active agent that modulates the TCA or oxidative phosphorylation.

11. The method of claim 1 wherein the disease or disorder is characterized by cells exhibiting a Warburg effect metabolic phenotype, and the active agent shifts the metabolism of the cells from glycolysis to glucose oxidation, reverses the suppression of mitochondria-dependent apoptosis, increase the oxidation of pyruvate, reduce the conversion of pyruvate to lactate, or a combination thereof; wherein the disease or disorder is characterized by cells exhibiting an Inverse Warburg effect metabolic phenotype, and the active agent shifts the metabolism of the cells from glucose oxidation to glycolysis, suppresses mitochondria-dependent apoptosis, decreases the oxidation of pyruvate, increases the conversion of pyruvate to lactate, or a combination thereof; or wherein disease or disorder is selected from the group consisting of a neurodegenerative disease or disorder, diabetes, a neurological disorder, seizure disorder, cardiovascular disease, ischemia, and endometriosis.

12. A method of monitoring the efficacy of a subject initially selected and treated according to the method of claim 1 comprising:
(a) comparing the level of hypoxia-inducible factor-1α (HIF-1α) or lactate in a control biological sample obtained from the subject before administration of one or more treatments to one or more treatment biological samples obtained after administration of the one or more treatments of the active agent, or
comparing the level of hypoxia-inducible factor-1α (HIF-1α) or lactate in a treatment biological samples obtained after administration of the one or more treatments of the active agent to a standard control, and
(b) adjusting the dosage or frequency of administration or discontinuing treatment with the active agent if the level of HIF-1α or lactate in at least one of the treatment biological samples is not increased or decreased compared the control biological sample.

13. The method of claim 12, wherein active agent is one that reverses the Warburg effect and dosage or frequency of administration is adjusted or discontinued if the level of HIF-1α or lactate in the treatment biological samples is not decreased compared the control biological sample such as a pyruvate dehydrogenase kinase inhibitor; or wherein active agent is one that reverses the Inverse Warburg effect and dosage or frequency of administration is adjusted or discontinued if the level of HIF-1α or lactate in the treatment biological samples is not increased compared the control biological sample.

14. The method of claim 13, wherein the biological samples are extracellular biological samples such as serum samples.

15. The method of any claim 12, wherein treatment is discontinued if the level of HIF-1α or lactate is not increased or decreased relative to control in a treatment biological sample obtained after at least 2, 3, 4, 5, or more administrations the active agent.

16. A method of monitoring the efficacy of a subject is initially selected and treated according to the method of claim 1 comprising:
(a) administering the subject Fluorodeoxyglucose (18F) and measuring its uptake in the diseased or disordered cells prior and after treatment with the active agent;
(b) adjusting the dosage or frequency of administration or discontinuing treatment with the active agent if the Fluorodeoxyglucose (18F) uptake in the diseased or disordered cells is not increased or decreased after treatment with the active agent.

17. The method of claim 16, wherein active agent is one that reverses the Warburg effect and dosage or frequency of administration is adjusted or discontinued if the Fluorodeoxyglucose (18F) uptake in the diseased or disordered cells is not decreased after treatment with the active agent such as a pyruvate dehydrogenase kinase inhibitor.

18. The method of claim 16, wherein active agent is one that reverses the Inverse Warburg effect and dosage or frequency of administration is adjusted or discontinued if the Fluorodeoxyglucose (18F) uptake in the diseased or disordered cells is not increased after treatment with the active agent, optionally wherein treatment is discontinued if the Fluorodeoxyglucose (18F) uptake in the diseased or disordered cells is not increased or decreased after at least 2, 3, 4, 5, or more administrations the active agent.

19. The method of claim 1, wherein the active agent is dichloroacetate (DCA) or an analogue, derivative, or conjugate thereof, further comprising
(a) comparing the level the active agent, maleylacetoacetate, maleylacetone, or delta-aminolevulinate in a biological sample obtained from the subject after treatment begins, and
(b) adjusting the dosage or frequency of administration or discontinuing treatment with the active agent if the level the active agent, maleylacetoacetate, maleylacetone, or delta-aminolevulinate is above a threshold indicting hepatotoxicity or neurotoxicity.

20. The method of claim 19, wherein the level of maleylacetone is compared in step (a), wherein the biological sample is urine.

21. A method of selecting and treating a subject for a disease or disorder comprising:
(a) detecting the level of MPC1, MPC2, or a combination thereof in diseased or disordered cells obtained from the subject;
(b) selecting the subject for treatment if the level of MPC1, MPC2, or the combination thereof is reduced relative to a control; and
(c) administering the selected subjects an effective amount of an inhibitor of citrate transporter or another transporter or enzyme related to formation or cycling of malate, citrate, or acetyl-CoA; or an inhibitor of glutaminolysis or a transporter or enzyme associated therewith such as glutaminase, to treat the disease or disorder.

22. A method of selecting and treating a subject for a disease or disorder comprising:
(a) detecting the level of HIF1, HIF2, or a combination thereof in diseased or disordered cells obtained from the subject that express wildtype, near wildtype, or not substantially reduced MPC1 or MPC2, or the combination thereof;
(b) selecting the subject for treatment if the level of HIF1, HIF2, or the combination thereof is not reduced or is increased relative to a control; and
(c) administering the selected subjects an effective amount of an inhibitor of glutaminolysis or a transporter or enzyme associated therewith such as glutaminase, to treat the disease or disorder.

23. A method of selecting and treating a subject for a disease or disorder comprising:
(a) detecting the level of MPC1, MPC2, or a combination thereof in diseased or disordered cells obtained from the subject;
(b) detecting the level of HIF1, HIF2, or a combination thereof in diseased or disordered cells obtained from the subject if the level of MPC1, MPC2, or the combination thereof is increased, the same, similar, or otherwise not substantially reduced relative to a control;
(c) selecting the subject for treatment if the level of HIF1, HIF2, or the combination thereof is not reduced or is increased relative to a control; and
(d) administering the selected subjects an effective amount of an inhibitor of glutaminolysis or a transporter or enzyme associated therewith such as glutaminase, to treat the disease or disorder,
Optionally wherein the inhibitor is selected from the group consisting of 4-chloro-3-{[(3-nitrophenyl)amino]sulfonyl}benzoic acid, BMS 303141, MEDICA 16, SB 204990, BPTES, CB-839, 968, EGCG, AG-120, and AG-221.


SEQUENCE LISTING (5 sequences)

SEQ ID NO: 1 - protein, 109 aa, Homo sapiens
MAGALVRKAADYVRSKDFRDYLMSTHFWGPVANWGLPIAAINDMKKSPEIISGRMTFALCCYSLTFMRFAYKVQPRNWLLFACHATNEVAQLIQGGRLIKHEMTKTASA

SEQ ID NO: 2 - DNA, 988 bp, Homo sapiens
gtcgtgaggcgggccttcgggctggctcgccgtcggctgccggggggttggcctgggtgtcattggctctgggaagcggcagcagaggcagggaccactcggggtctggtgtcggcacagccatggcgggcgcgttggtgcggaaagcggcggactatgtccgaagcaaggatttccgggactacctcatgagtacgcacttctggggcccagtagccaactggggtcttcccattgctgccatcaatgatatgaaaaagtctccagagattatcagtgggcggatgacatttgccctctgttgctattctttgacattcatgagatttgcctacaaggtacagcctcggaactggcttctgtttgcatgccacgcaacaaatgaagtagcccagctcatccagggagggcggcttatcaaacacgagatgactaaaacggcatctgcataacaatgggaaaaggaagaacaaggtcttgaagggacagcattgccagctgctgctgagtcacagatttcattataaatagcctccctaaggaaaatacactgaatgctatttttactaaccattctatttttatagaaatagctgagagtttctaaaccaactctctgctgccttacaagtattaaatattttacttctttccataaagagtagctcaaaatatgcaattaatttaataatttctgatgatgttttatctgcagtaatatgtatatcatctattagaatttacttaatgaaaaactgaagagaacaaaatttgtaaccactagcacttaagtactcctgattcttaacattgtctttaatgaccacaagacaaccaacagctggccacgtacttaaaattttgtccccactgtttaaaaatgttacctgtgtatttccatgcagtgtatatattgagatgctgtaacttaatggcaataaatgatttaaatatttgttaaaaaaaaaaaaaaaaaaaaaaaaaaaaa

SEQ ID NO: 3 - protein, 127 aa, Homo sapiens [sequence omitted]

SEQ ID NO: 4 - DNA, 798 bp, Homo sapiens
ctcagcgcctccgccccggggcccccgctcacccaggtatcgactccgcagccgggacgggtcctccagcccgagggaccttttcctcacgtcccacaacagccagggacgagaacacagccacgctcccacccggctgccaacgatccctcggcggcgatgtcggccgccggtgcccgaggcctgcgggccacctaccaccggctcctcgataaagtggagctgatgctgcccgagaaattgaggccgttgtacaaccatccagcaggtcccagaacagttttcttctgggctccaattatgaaatgggggttggtgtgtgctggattggctgatatggccagacctgcagaaaaacttagcacagctcaatctgctgttttgatggctacagggtttatttggtcaagatactcacttgtaattattccaaaaaattggagtctgtttgctgttaatttctttgtgggggcagcaggagcctctcagctttttcgtatttggagatataaccaagaactaaaagctaaagcacacaaataaaagagttcctgatcacctgaacaatctagatgtggacaaaaccattgggacctagtttattatttggttattgataaagcaaagctaactgtgtgtttagaaggcactgtaactggtagctagttcttgattcaatagaaaaatgcagcaaacttttaataacagtctctctacatgacttaaggaacttatctatggatattagtaacatttttctaccatttgtccgtaataaaccatacttgctcgtatata

SEQ ID NO: 5 - protein, 216 aa, Homo sapiens
MQAGKPILYSYFRSSCSWRVRIALALKGIDYKTVPINLIKDRGQQFSKDFQALNPMKQVPTLKIDGITIHQSLAIIEYLEEMRPTPRLLPQDPKKRASVRMISDLIAGGIQPLQNLSVLKQVGEEMQLTWAQNAITCGFNALEQILQSTAGIYCVGDEVTMADLCLVPQVANAERFKVDLTPYPTISSINKRLLVLEAFQVSHPCRQPDTPTELRA